(19) United States
(12) Patent Application Publication
(10) Pub. No.: US 2023/0293438 A1
(43) Pub. Date: Sep. 21, 2023

(54) INJECTABLE DRUG-RELEASING MICROPOROUS ANNEALED PARTICLE SCAFFOLDS FOR TREATING MYOCARDIAL INFARCTION

(71) Applicant: THE REGENTS OF THE UNIVERSITY OF CALIFORNIA, Oakland, CA (US)

(72) Inventors: Jun Fang, Los Angeles, CA (US); Song Li, Los Angeles, CA (US); Dino Di Carlo, Los Angeles, CA (US); Jaekyung Koh, Los Angeles, CA (US)

(73) Assignee: THE REGENTS OF THE UNIVERSITY OF CALIFORNIA, Oakland, CA (US)

(21) Appl. No.: 18/015,863

(22) PCT Filed: Jul. 15, 2021
(86) PCT No.: PCT/US2021/041888

§ 371 Date: Jan. 12, 2023

Related U.S. Application Data
(60) Provisional application No. 63/052,841, filed on Jul. 16, 2020.

(51) Int. Cl. A61K 9/16; A61K 9/06; A61K 31/352; A61K 31/444; A61P 9/10; A61K 47/10

(52) U.S. Cl. CPC A61K 9/167; A61K 9/06; A61K 9/1647; A61K 31/352; A61K 31/444; A61K 47/10; A61P 9/10

(57) ABSTRACT

A therapeutic microporous hydrogel scaffold for use in an animal is disclosed that releases one or more therapeutic agents or drugs. The scaffold uses a drug-releasing microporous annealed particle system that encapsulates drug-loaded nanoparticles into particle building blocks. By modulating nanoparticle hydrophilicity and pre-gel solution viscosity, the particle building blocks were generated with consistent and homogeneous encapsulation of nanoparticles. The scaffold may be used to treat myocardial infarction (MI) using, for example, the drugs forskolin (F) and Repsox (R). The intramyocardial injection of the pre-annealed hydrogel slurry of particles that formed the resultant scaffold improved left ventricular functions, which were further enhanced with increased angiogenesis and reduced fibrosis and inflammatory response. This therapeutic microporous hydrogel scaffold platform represents a new generation of microgel particles for MI therapy and will have broad applications in regenerative medicine and disease therapy.

### RELATED APPLICATION

This Application claims priority to U.S. Provisional Pat. Application No. 63/052,841 filed on Jul. 16, 2020, which is hereby incorporated by reference. Priority is claimed pursuant to 35 U.S.C. § 119 and any other applicable statute.

### TECHNICAL FIELD

The technical field generally relates to injectable hydrogels used to treat myocardial infarction (MI). More specifically, the technical field relates to a microporous hydrogel scaffold formed from annealed particles. The particles that are used to form the scaffold structure encapsulate nanoparticles loaded with one or more drugs.

### STATEMENT REGARDING FEDERALLY SPONSORED RESEARCH AND DEVELOPMENT

This invention was made with government support under Grant Number HL121450, awarded by the National Institutes of Health, and Grant Number N00014-16-1-2997 awarded by the Office of Naval Research. The government has certain rights in the invention.

### BACKGROUND

Ischemic heart disease (IHD) is a leading cause of global mortality, accounting for over nine million deaths per year, according to the World Health Organization (WHO). Acute MI is the most common manifestation of IHD, usually caused by the complete occlusion of a coronary artery with atherosclerotic plaque rupture and thrombosis. Following MI, the damaged myocardium eventually undergoes a remodeling process with cardiomyocyte depletion, tissue fibrosis, cardiac dilatation and dysfunction, culminating in heart failure. Currently, several therapeutic strategies have been exploited to repair and regenerate the damaged cardiac tissues caused by MI, including pharmaceutic approaches, injectable hydrogels, cardiac patches, cell transplantation, and cell reprogramming. Among them, injectable hydrogels have shown great potential to treat MI by providing mechanical support and tissue integration to increase myocardial thickness and prevent ventricular remodeling through a minimally invasive and cost-effective manner. Nevertheless, traditional hydrogels usually have a trade-off between mechanical strength to support cell attachment and porous structure to enable rapid tissue ingrowth before hydrogel degradation. Thus, biomaterials with independently tunable biophysical properties are needed to improve therapeutic outcomes.

Mechanical properties, porosity and microarchitecture of porous hydrogels can significantly impact in vivo cell behavior and tissue regeneration effects. Currently, a variety of manufacturing techniques have been developed to fabricate structured porous hydrogel scaffolds, including solvent/porogen leaching, gas foaming, freeze-drying and 3D printing, but these methods are challenging to be delivered via minimally invasive techniques. To decouple porous structure and mechanical support, an injectable microporous annealed particle (MAP) scaffold has been developed by crosslinking uniform microgel (µGel) building blocks produced in a microfluidic device. For example, International Patent Application Publication No. WO 2016/011387, which is incorporated herein by reference, discloses an example of such MAP scaffolds. By combining injectability, microporosity and mechanical strength, the porous MAP scaffolds have demonstrated rapid cellular infiltration without bulk material degradation to facilitate wound and stroke healing in vivo. However, the therapeutic efficacy of MAP gel for treating MI and its capabilities as a drug delivery platform to promote functional regeneration remain to be investigated.

Pharmacological treatments are commonly used in clinic to slow down or reverse detrimental cardiac remodeling in MI patients, with specific effects such as pro-angiogenesis, anti-fibrosis, anti-inflammatory, anti-cardiomyocyte death, antiarrhythmic, and anti-thrombosis. The cyclic adenosine monophosphate (cAMP) is an essential second messenger and mediates many critical intracellular signaling under physiological and pathophysiological conditions. Activation and increased generation of cAMP can markedly increase cardiac LV function and survival, and attenuate cardiac fibrosis and its sequelae after acute MI. Additionally, transforming growth factor β (TGF-β) signaling plays a pleiotropic role in driving disease progression. TGF-β expression is upregulated in acute MI and cardiac hypertrophy, which leads to fibrosis and diastolic dysfunction with induced myo-differentiation, extracellular matrix (ECM) synthesis, and cardiomyocyte hypertrophy.

### SUMMARY

In one embodiment, a drug-releasing annealed microparticle system is disclosed (sometimes referred to herein as drugMAP), which has been developed by encapsulating hydrophobic drug-loaded nanoparticles into the microgel particle building blocks via microfluidic manufacturing. The particle building blocks can be generated with consistent and homogeneous encapsulation of nanoparticles by modulating nanoparticle hydrophilicity and pre-gel solution viscosity. In one preferred embodiment, the use of PLGA55k-b-PEG5k nanoparticles and the addition of hyaluronic acid (HA) into the pre-gel solution increased the stability of nanoparticle suspension and delayed the particle aggregation in the microfluidic channel. The microfluidic production enabled highly monodisperse microgel particles of well-defined size as well as the controllable amount of nanoparticles in each microgel.

One or more drugs may be loaded into the nanoparticles. In one specific embodiment, two (2) hydrophobic drugs of forskolin (F, a cyclic adenosine monophosphate (cAMP) activator) and Repsox (R, a transforming growth factor-β (TGF-β) inhibitor) are loaded into PLGA-based nanoparticles to make the therapeutic microporous hydrogel scaffold, and demonstrate the additive and complementary effects on promoting cardiomyocyte survival, inhibiting fibroblast myo-differentiation and enhancing endothelial cells proliferation and angiogenesis in vitro. The microporous hydrogel scaffold can be injected into heart tissue for MI therapy by endowing pleiotropic benefits with providing mechanical support, promoting cell migration and neovascularization, suppressing fibrosis and modulating immune responses.

The therapeutic microporous hydrogel scaffold may be injected directly into the heart tissue using a delivery device. For example, a catheter-like device may be used to inject the unannealed hydrogel slurry through the internal chambers of the heart and into the cardiac tissue which is then annealed in situ as explained herein. Alternatively, the external tissues of the heart muscle may be injected with the therapeutic microporous hydrogel scaffold using an endoscopic or laparoscopic device. A small incision in the chest or other access points may be used to insert the delivery device. The hydrogel slurry may be injected by using one or more needles on the delivery device. In one embodiment, the spherical hydrogel particles may anneal in response to an endogenous annealing agent (e.g., factor XIIIa (FXIIIa)). Alternatively, the spherical hydrogel particles may anneal to one another in response to an exogenous annealing agent that is added to the hydrogel slurry. This may be delivered by the same or different delivery device that delivers the hydrogel pre-annealed slurry of particles.

In one embodiment, a therapeutic hydrogel system for use in an animal to treat MI includes a plurality of spherical hydrogel particles decorated with K peptides and Q peptides and having distributed therein a plurality of nanoparticles loaded with one or more therapeutic agents or drugs, wherein the plurality of spherical hydrogel particles when exposed to an annealing agent, induces surface binding between the spherical hydrogel particles via the K peptides and Q peptides and forms a porous scaffold.

In another embodiment, a therapeutic hydrogel system for use in an animal includes a plurality of spherical hydrogel particles having distributed therein a plurality of nanoparticles loaded with one or more therapeutic agents or drugs, wherein the plurality of spherical hydrogel particles when exposed to an annealing agent, induces surface binding between the spherical hydrogel particles and forms a porous scaffold. The therapeutic hydrogel system may also include an exogenous annealing agent that is delivered with or exposed to the hydrogel particles to create the porous scaffold.

In another embodiment, a method of manufacturing a therapeutic hydrogel includes providing a microfluidic device configured to generate aqueous emulsions in an oil phase, the microfluidic device having a first aqueous phase microfluidic channel and a second aqueous phase microfluidic channel interfacing in an emulsion generating region, the emulsion generating region interfacing with a first oil phase microfluidic channel and a second oil phase microfluidic channel; flowing a first aqueous solution into the first aqueous phase microfluidic channel comprising PEG vinyl sulfone pre-reacted with K-peptide, Q-peptide, and a plurality of nanoparticles loaded with one or more therapeutic agents or drugs; flowing a second aqueous solution into the second aqueous phase microfluidic channel comprising a MMP-sensitive crosslinker; flowing oil into the first and second oil phase microfluidic channels to generate emulsions of the mixed solution of the first and second aqueous solutions in the oil; and allowing the emulsions to crosslink to form spherical hydrogel particles.

In another embodiment, a method of treating myocardial infarction in an animal includes injecting a pre-annealed therapeutic hydrogel slurry (e.g., therapeutic hydrogel system) into heart tissue of the animal, the pre-annealed therapeutic hydrogel slurry includes a plurality of spherical hydrogel particles decorated with K peptides and Q peptides and having distributed therein a plurality of nanoparticles loaded with one or more therapeutic agents or drugs, wherein the plurality of spherical hydrogel particles when exposed to an annealing agent, induces surface binding between the spherical hydrogel particles via the K peptides and Q peptides and forms a porous scaffold. The one or more therapeutic agents or drugs may include a cAMP agonist and/or a TGF-β inhibitor. As one particular example, the one or more therapeutic agents or drugs comprises Forskolin and 2-(3-(6-Methylpyridine-2-yl)-1H-pyrazol-4-yl)-1,5-naphthyridine (RepSox).

### BRIEF DESCRIPTION OF THE DRAWINGS

FIG. 1A illustrates a microporous hydrogel scaffold formed from annealed particles for MI therapy. The particles anneal to form the microporous hydrogel scaffold that contains nanoparticles loaded with one or more therapeutic agents or drugs.

FIG. 1B schematically illustrates the microfluidic generation of drug-releasing a microporous hydrogel scaffold formed from annealed particles for MI therapy. (FIG. 1A) Microfluidic generation of the particle building blocks was done by encapsulating drug/nanoparticles into micrometer sized beads or spheres to generate particles containing nanoparticles loaded with a drug or therapeutic agent in a microfluidic device. The spherical hydrogel particles are formed by crosslinking pre-gel solutions via thiol-ene reactions to encapsulate nanoparticles in the gel mesh.

FIG. 1C schematically illustrates the injection of the of pre-annealed hydrogel slurry that forms the cardiac microporous hydrogel scaffold for MI therapy. The delivery of specific drugs contained in the microporous hydrogel scaffold endows the scaffold with pleiotropic benefits for heart repair.

FIG. 2A illustrates the microfluidic channel design for generating the spherical hydrogel particles that are used in the microporous hydrogel scaffold. Illustrated features include the oil inlets, aqueous inlets, the droplet generation region, and the droplet collection region.

FIG. 2B is a photograph of the microfluidic device that includes the microfluidic channel design of FIG. 2A, channels are highlighted with colored dye solutions.

FIG. 2C illustrates an image of PEG-VS pre-gel solution with dispersed nanoparticles (NPs) flows stably through the inlet filters. Insert image in the lower-left corner is a representative SEM image of PLGA-based NPs.

FIG. 2D illustrates the formation of homogeneous droplets containing pre-gel solution and crosslinker formed at a flow-focusing junction of the microfluidic channel.

FIG. 2E illustrates an image of nanoparticle-spherical particles or bead structures (i.e., NPs-µGels) with a uniform nanoparticle distribution collected at the outlet region.

FIG. 2F shows fluorescence images of droplets generated with fluorescent-labeled aqueous solutions, one aqueous channel with coumarin-6 (left) labeled NPs with 4-arm PEG-VS pre-gel solution and another aqueous channel with AF 546-maleimide (middle) with MMP-sensitive crosslinker solution. The right image shows the T-junction and formation of the droplets.

FIG. 2G shows representative fluorescent images of NPs-µGel beads made under optimized processing conditions, with NPs distributed uniformly in the particles/beads or µGels.

FIG. 3A illustrates a graph showing how the generation of NPs-µGel beads with highly defined sizes is possible by altering the aqueous flow rate.

FIG. 3B illustrates the diameter of the NPs-µGel beads, made with an aqueous flow rate of 8 uL/min, and swollen in the buffer after aqueous extraction from the oil phase. Qᵥ represents the volumetric swelling ratio of a bead.

FIG. 3C shows representative images of NPs-µGel beads loaded with increasing amounts of NPs. The numbers in brackets represent the weight percentages of the NPs to dry pre-gel components.

FIG. 3D is a graph showing nanoparticle loading efficiency in different NP-µGel beads as a function of wt%.

FIG. 3E is a graph showing the nanoparticle loading concentration in NPs-µGel beads as a function of initial concentration.

FIG. 3F illustrates microporous hydrogel scaffolds generated by annealing NPs-µGel beads using FXIIIa.

FIG. 3G illustrates graphs showing pore size (left) and void fraction (right) of conventional MAP scaffolds and microporous hydrogel scaffolds formed from annealed particles for MI therapy.

FIG. 3H illustrates the storage moduli of bulk hydrogels mixed with different amounts of NPs. Data are shown as mean ± SD. *p < 0.05, NS represents no significant difference.

FIG. 4A illustrates a table showing the summarized drug effects of forskolin (F), Repsox (R), and FR on various cardiac remodeling-associated cells. Sign + represents a positive effect, and sign – represents a negative effect.

FIG. 4B is a graph showing the cumulative drug release profiles from FR/NPs (FR loaded NPs) and FR/therapeutic microporous hydrogel which contain the one or more therapeutic agents or drugs (also referred to in the drawings as FR/drugMAP) (F and R loaded microporous hydrogel scaffolds).

FIG. 4C shows live and dead staining of neonatal cardiomyocytes cultured in the indicated conditions on day 3.

FIG. 4D shows a graph of cell viability of neonatal cardiomyocytes.

FIG. 4E illustrates myo-differentiation of neonatal cardiac fibroblasts cultured in the indicated conditions on day 5.

FIG. 4F shows mean fluorescent intensity of α-SMA in FIG. 4E.

FIG. 4G shows representative fluorescent images of vascular network formation. Human umbilical vein endothelial cells (HUVECs) are cultured at the indicated conditions for 16 hours and stained with Calcein-AM.

FIG. 4H illustrates the quantification of junction numbers (left), tube numbers (middle) and mesh numbers (right). Data are shown as mean ± SD. *p < 0.05 and **p < 0.01 indicate comparisons to blank. ## p < 0.01 indicates comparisons to R condition. NS represents no significant difference.

FIG. 5A: illustrates representative Masson’s trichrome-stained sections of infarcted rat hearts after 5 weeks treatment with PBS, FR/NPs, MAP gel and FR/ microporous hydrogel scaffold (FR/drugMAP gel). High-magnification views of the infarcted zones are presented below image cross-sectional image.

FIG. 5B illustrates plots showing the quantitative analyses of infarcted size (as % of the total LV area).

FIG. 5C illustrates plots showing the quantitative analyses of infarcted minimum LV wall thickness.

FIG. 5D illustrates plots of LVEDV of infarcted hearts measured by echocardiography at 5 weeks.

FIG. 5E illustrates plots of LVESV of infarcted hearts measured by echocardiography at 5 weeks.

FIG. 5F illustrates LV ejection fraction (EF) of infarcted hearts at day 2 (baseline) and week 5 after treatment.

FIG. 5G illustrates the change in LVEF in comparison to baseline (ΔLVEF). Data are shown as mean ± SD. PBS (n = 9), FR/NPs (n = 6), MAP (n = 9) and FR/drugMAP gel (n = 9).*p < 0.05 and **p < 0.01 indicate significant difference in comparison to the PBS control group. #p < 0.05 and ## p < 0.01 in (FIG. 5F) indicate comparisons of 5 week treated group to the corresponding baseline. NS represents no significant difference.

FIG. 6A shows representative images of angiogenesis staining with α-SMA and vWF in the central infarct LV zone of hearts treated with PBS, FR/NPs, MAP and FR/drugMAP gel at 5 weeks. Microgel beads were labeled by AF546 dye for material tracking.

FIG. 6B shows representative images of macrophage staining with CD68.

FIG. 6C illustrates the quantification of capillary density (vWF+ vessels) of the central infarct LV zone of hearts treated with PBS (n =9), FR/NPs (n =6), MAP (n =9) and FR/drugMAP (n = 9) at 5 weeks. Data are shown as mean ± SD. *p < 0.05 and **p < 0.01 indicate significant difference in comparison to PBS control group.

FIG. 6D shows the arteriolar density (α-SMA+ vessels) of the central infarct LV zone of hearts treated with PBS (n =9), FR/NPs (n =6), MAP (n =9) and FR/drugMAP (n = 9) at 5 weeks.

FIG. 6E shows the macrophage density in the central infarct LV zone of hearts treated with PBS (n =9), FR/NPs (n =6), MAP (n =9) and FR/drugMAP (n = 9) at 5 weeks.

FIG. 7A illustrates the optimized preparation of NPs-µGel building blocks. NP aggregation and sedimentation can lead to the failure in NPs-µGel preparation. Two strategies, including improving NP hydrophilicity via PEG surface modification and increased pre-gel solution viscosity via the addition of hyaluronic acid (HA), were applied to prevent or delay NP aggregation and sedimentation to achieve stable preparation of NPs-µGel beads with uniform particle distribution (see rightmost image).

FIG. 7B illustrates fluorescence microscopy images of NPs-µGel building blocks. NPs were labeled by coumarin-6 dye, µGels were labeled by AF546 dye.

FIGS. 8A-8B: Hydrogel degradation and model drug release profile from the drug-containing microporous hydrogel scaffold. FIG. 8A shows Alexa Fluor 546 and Coumarin-6 release as surrogates for hydrogel degradation and drug release from hydrogels after a 3-day incubation in PBS and collagenase solution at the different concentrations. Data are shown as mean ± SD, n = 4. FIG. 8B shows representative fluorescent images of particles pushed through a 110 × 110 µm microfluidic channel following a 3-day degradation in PBS and collagenase solutions showing the hydrogel and drug remnant as well as the hydrogel deformability.

FIG. 9 shows the drug effects on cardiomyocyte viability. Live/dead staining images of neonatal rat cardiomyocytes cultured with small molecule drugs contained in the medium. F represents forskolin, R represents Repsox, FR represents the combination of F and R.

FIGS. 10A-10B show the drug effects on cardiomyocyte proliferation. FIG. 10A includes fluorescent images of neonatal cardiomyocytes cultured in the indicated conditions on day 1. FIG. 10B shows a graph of the cell proliferation rate is calculated based on the fraction of cells positive with EdU in FIG. 10A. Data are shown as mean ± SD. **p < 0.01.

FIGS. 11A-11B: Drug effects on cardiac fibroblast proliferation and myo-differentiation. FIG. 11A shows MTS assay results of fibroblast proliferation. Data are shown as mean ± SD. *p < 0.05, **p < 0.01. FIG. 11B illustrates myo-differentiation of cardiac fibroblasts in the indicated conditions at day 5. NP-s represents nanoparticle supernatant. MAP-s represents MAP gel supernatant.

FIGS. 12A-12B: Drug effects on endothelial cell proliferation and tubule network formation. FIG. 12A MTS assay results of endothelial cell proliferation. FIG. 12B shows representative fluorescent images of vascular network formation. Human umbilical vein endothelial cells (HUVECs) are cultured at the indicated conditions for 16 hours and stained with Calcein-AM. VEGF at 20 ng/mL is used as a positive control. Data are shown as mean ± SD. *p < 0.05, **p < 0.01.

FIGS. 13A-13B illustrate that the drug-containing microporous hydrogel scaffold prevents cellular uptake of NPs. FIG. 13A shows fibroblast cellular uptake of NPs after 1 day and 4 days contacting culture with free NPs or NPs-µGel, green: coumarin-6 labeled NPs, red: phalloidin F-actin. FIG. 13B shows fluorescent intensity of NPs in the area of cells in FIG. 13A. Data are shown as mean ± SD. **p < 0.01.

FIG. 14A is a schematic overview of rat heart slices from the area below the ligature.

FIG. 14B shows Masson’s trichrome staining of slides of left ventricular infarcted rats treated with PBS, FR/NPs, MAP gel and FR/drugMAP gel for 5 weeks.

FIG. 15 illustrate images showing how microporous hydrogel scaffolds (i.e., drugMAP scaffolds) enhance in vivo neovascularization in MI therapy. Neovascularization in the central infarct LV zone of hearts treated with PBS, FR/NPs, MAP and FR/drugMAP gel at 5 weeks. Microgel beads were labeled by AF546 dye for material tracking. High-magnification views of the white dashed border areas in the upper images are shown below the respective images.

FIG. 16 illustrate images showing how microporous hydrogel scaffolds (i.e., drugMAP scaffolds) scaffolds reduce the immune response in MI therapy. Macrophage staining with CD68 in infarcted hearts treated with PBS, FR/NPs, MAP and FR/drugMAP gel for 5 weeks.

FIG. 17 illustrates one embodiment of a delivery device that may be used to deliver the therapeutic microporous hydrogel scaffold to a human (e.g., mammalian) heart. The delivery device may be inserted intravascularly or endoscopically/laparoscopically.

### DETAILED DESCRIPTION OF THE ILLUSTRATED EMBODIMENTS

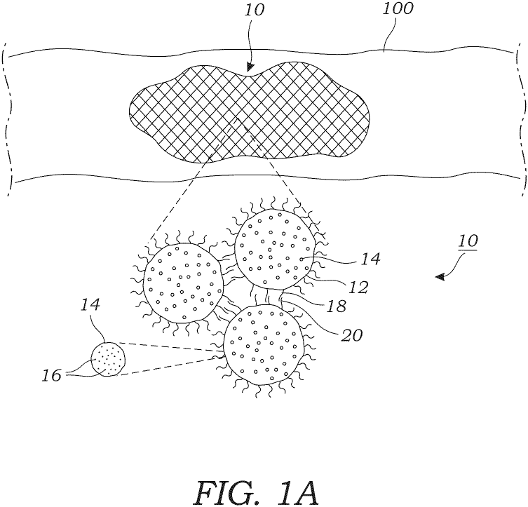

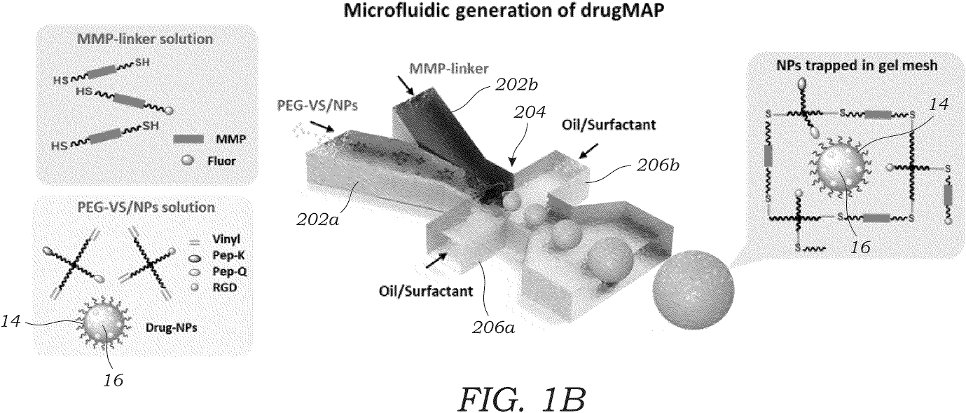

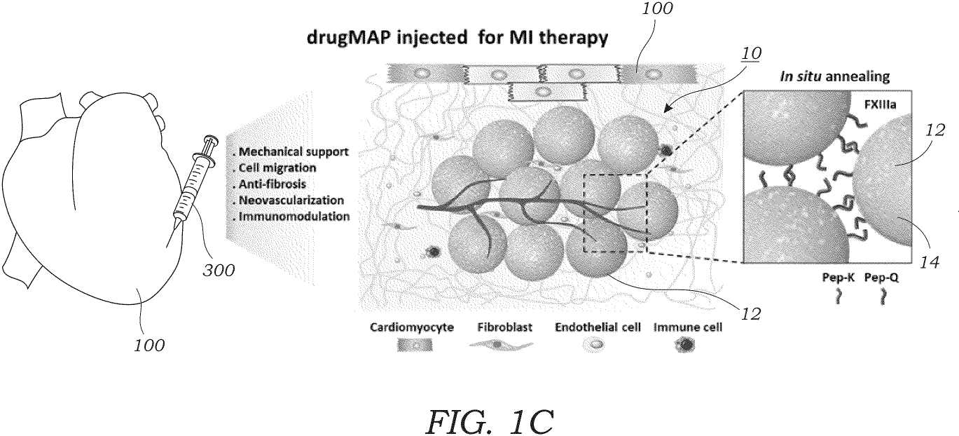

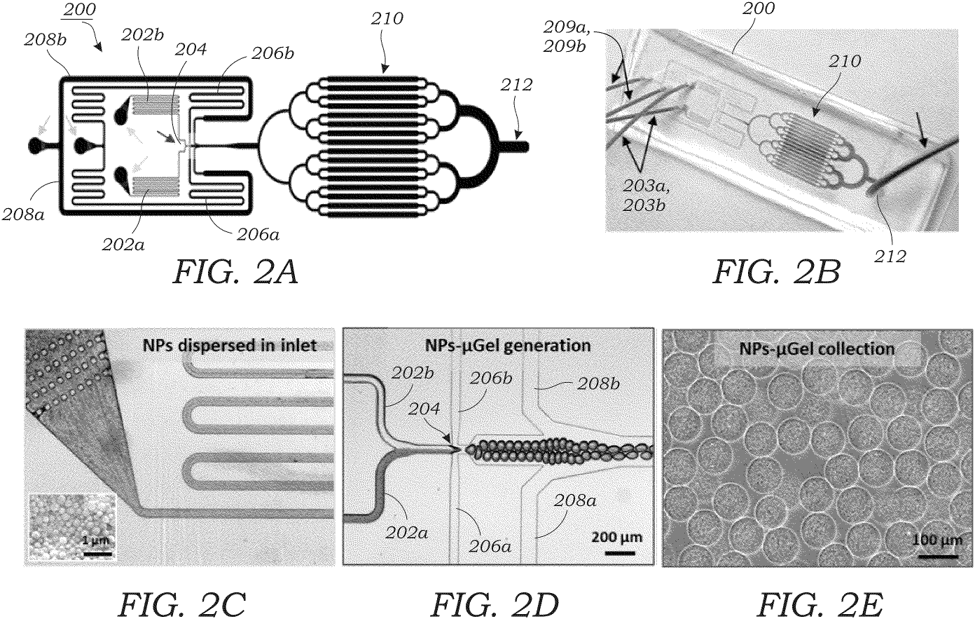

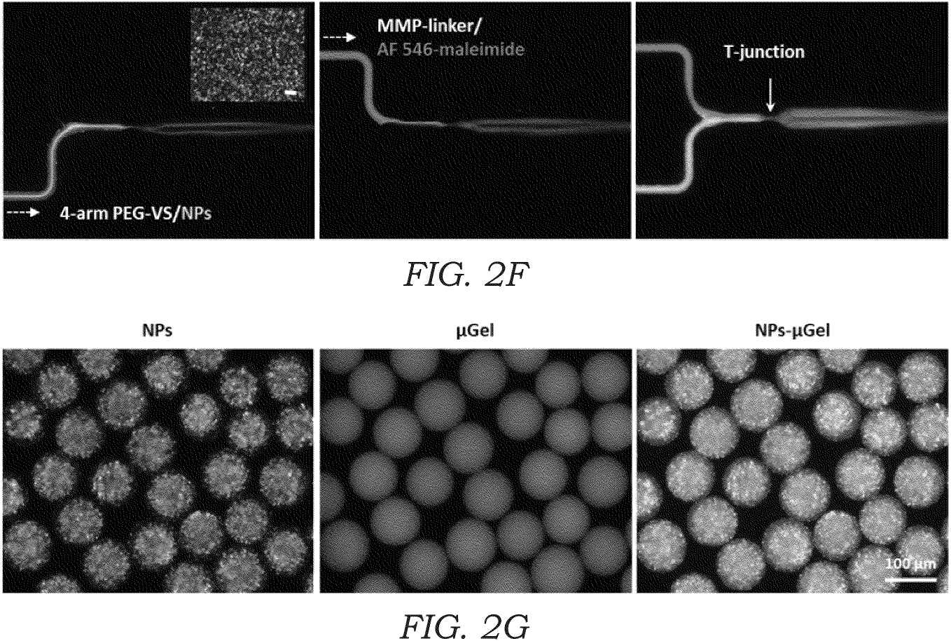

In one embodiment, a therapeutic microporous hydrogel scaffold 10 is disclosed that is used in animal heart tissue 100 (e.g., mammalian heart tissue) to treat MI. FIG. 1A illustrates one such embodiment of a therapeutic microporous hydrogel scaffold 10 that forms in animal heart tissue 100. The therapeutic microporous hydrogel scaffold 10 is formed from spherical hydrogel particles 12 that act as “building blocks” that, in one embodiment, are generated using a microfluidic device 200 as seen in FIGS. 1B, 2A and explained below. The spherical hydrogel particles 12 anneal to one another in situ on and/or within the heart tissue 100 (or other tissue 100) using an endogenous and/or exogenous annealing agent to form the therapeutic microporous hydrogel scaffold 10. The therapeutic microporous hydrogel scaffold 10 includes micrometer-sized pores therein. The size of the pores contained therein allows for the intrusion and migration of cells into the therapeutic microporous hydrogel scaffold 10 from the surrounding tissue 100. Typical pore sizes may include those about 10 µm or greater. For example, pore sizes between about 10 µm to about 50 µm may be preferred in some embodiments.

The spherical hydrogel particles 12 contain encapsulated nanoparticles 14 that are loaded with one or more therapeutic agents or drugs 16. In some embodiments such as those described herein, the therapeutic agent or drug 16 is hydrophobic. In other embodiments, the therapeutic agent or drug 16 is hydrophilic. The therapeutic agent or drug 16 may also include small molecules, cytokines, proteins/peptides or fragments thereof, vaccines, nucleic acids, genes or genetic sequences, biomolecules, and the like. The nanoparticles 14 may be made by diverse biodegradable synthetic and natural polymers. Natural polymers include polysaccharides (chitosan, hyaluronic acid, dextran), and proteins (collagen, gelatin, elastin). Biodegradable synthetic polymers include poly(lactic acid) (PLA), poly(glycolic acid) (PGA), polycaprolactone (PCL), polyhydroxyalkanoates (PHA), and their copolymers, poly(ethylene glycol) (PEG) containing polyesters (PLGA-mPEG, PLA-PEG-PLA), polyurethanes (PU), polyamides (polylysine, polyglutamic acid), polyanhydrides, etc. In one preferred embodiment for the particular application to treat MI, the nanoparticles 14 are made from a biodegradable polyester material such as poly(lactic-co-glycolic acid) (PLGA) based polymers. This includes PLGA 35k, and different PLGA-PEG copolymers, such as poly(ethylene glycol) methyl ether-block-poly(lactide-co-glycolide) (e.g., PLGA55k-b-PEG5k, and PLGA25k-b-PEG5k). The nanoparticles 14 may be formed from other polymers known for drug delivery. This includes hydrophobic polymers that can carry therapeutic agents.

The collection of spherical hydrogel particles 12 that contain the drug-containing nanoparticles 14 form a hydrogel system that is delivered to the tissue 100 via a delivery device 300 such as that described in further detail herein. The spherical hydrogel particles 12 are linked or annealed to one another, for example, using peptides 18, 20 (e.g., K peptide 18 and Q peptide 20) that are populated or decorated on the spherical hydrogel particles 12 as seen in FIG. 1A. Each hydrogel particle 12 has both types of peptides 18, 20 (e.g., K, Q) populated thereon. The spherical hydrogel particles 12 may also be populated or decorated with an optional cell adhesive moiety such as RGD peptide. The therapeutic microporous hydrogel scaffold 10 may be made to be biodegradable by incorporating MMP-sensitive peptides in the gel matrix of the spherical hydrogel particles 12, making it degradable by MMP enzyme. The collection of spherical hydrogel particles 12, in a pre-annealed state, form a slurry that can readily be delivered to the desired tissue location for annealing into the final scaffold 10 using the delivery device 300.

While peptides 18, 20 are disclosed as one example an annealing moiety used to link adjacent hydrogel particles 12 together to form the therapeutic microporous scaffold 10, it should be appreciated that hydrogel particles 12 may be linked using other chemistries. For example, radically-initiated polymerization may be used to connect hydrogel particles 12. This includes chemical-initiators such as ammonium persulfate combined with Tetramethylethylenediamine. Alternatively, photoinitators such as Irgacure® 2959 or Eosin Y together with a free radical transfer agent such as a free thiol group (used at a concentration within the range of 10 µM to 1 mM) may be used in combination with a light source that is used to initiate the reaction as described herein. One example of a free thiol group may include, for example, the amino acid cysteine, as described herein. Of course, peptides including a free cysteine or small molecules including a free thiol may also be used. Another example of a free radical transfer agent includes N-Vinylpyrrolidone (NVP).

Alternatively, Michael and pseudo-Michael addition reactions, including α,β-unsaturated carbonyl groups (e.g., acrylates, vinyl sulfones, maleimides, and the like) to a nucleophilic group (e.g., thiol, amine, aminoxy) may be used to hydrogel particles 12 to form the therapeutic microporous scaffold 10. In another alternative embodiment, hydrogel particle 12 formation chemistry allows for network formation through initiated sol-gel transitions including fibrinogen to fibrin (via addition of the catalytic enzyme thrombin).

Functionalities that allow for particle-particle annealing are included either during or after the formation of the hydrogel particles 12. In one or more embodiments, these functionalities include α,β-unsaturated carbonyl groups that can be activated for annealing through either radical initiated reaction with α,β-unsaturated carbonyl groups on adjacent hydrogel particles 12 or Michael and pseudo-Michael addition reactions with nucleophilic functionalities that are either presented exogenously as a multifunctional linker between particles or as functional groups present on adjacent hydrogel particles 12. This method can use multiple hydrogel particle 12 population types that when mixed form the therapeutic microporous scaffold 10. For example, a hydrogel particle 12 of type X presenting, for example, nucleophilic surface groups can be used with hydrogel particle 12 type Y presenting, for example, α,β-unsaturated carbonyl groups. In another embodiment, functionalities that participate in Click chemistry can be included allowing for attachment either directly to adjacent hydrogel particles 12 that present complimentary Click functionalities or via an exogenously presented multifunctional molecule that participates or initiates (e.g., copper) Click reactions. Another example of Click chemistry includes using a click reaction of reaction of norbornene groups at the surfaces of the hydrogel particles 12 (e.g., hyaluronic acid-norbornene hydrogel particles 12) and a separate 4-arm PEG-tetrazine (PEG-Tet) crosslinker).

The annealing functionality can include any previously discussed functionality used for hydrogel particle 12 crosslinking that is either orthogonal or similar (if potential reactive groups remain) in terms of its initiation conditions (e.g., temperature, light, pH) compared to the initial crosslinking reaction. For example, if the initial crosslinking reaction consists of a Michael-addition reaction that is temperature dependent, the subsequent annealing functionality can be initiated through temperature or photo initiation (e.g., Eosin Y, Irgacure®). As another example, the initial hydrogel particles 12 may be photopolymerized at one wavelength of light (e.g., ultraviolent with Irgacure®), and annealing of the hydrogel particles 12 to form the therapeutic microporous scaffold 10 occurs at the same or another wavelength of light (e.g., visible with Eosin Y) or vice versa. Besides annealing with covalent coupling reactions, annealing moieties can include non-covalent hydrophobic, guest/host interactions (e.g., cyclodextrin), hybridization between complementary nucleic acid sequences or nucleic acid mimics (e.g., protein nucleic acid) on adjoining hydrogel particles 12, or ionic interactions. An example of an ionic interaction would consist of alginate functionality on the hydrogel particle surfaces that are annealed with Ca2+. So-called “A+B” reactions can be used to anneal hydrogel particles 12 as well. In this embodiment, two separate hydrogel particle 12 types (type A and type B) are mixed in various ratios (between 0.01:1 and 1:100 A:B) and the surface functionalities of type A react with type B (and vice versa) to initiate annealing. These reaction types may fall under any of the mechanisms listed herein.

The spherical hydrogel particles 12 are formed by crosslinking pre-gel solutions via thiol-ene reactions to encapsulate the nanoparticles 14, which contain the one or more therapeutic agents or drugs 16 therein, in the gel mesh. The spherical hydrogel particles 12 are then annealed to one another in situ, at the site of injection, to form a three-dimensional therapeutic microporous hydrogel scaffold 10 for MI therapy. The formed scaffold 10 endows pleiotropic benefits for heart repair.

In one embodiment as explained herein, the therapeutic microporous hydrogel scaffold 10 is used in heart tissue 100 to promote cardiac regeneration by activating cAMP pathway while inhibiting TGF-β signaling. Forskolin (F) (CAS Registry No. 66575-29-9) is a cAMP agonist, and 2-(3-(6-Methylpyridine-2-yl)-1H-pyrazol-4-yl)-1,5-naphthyridine or RepSox (R) is a selective TGF-β inhibitor. Both small molecules have shown the beneficial effects to rescue cardiac dysfunction and ameliorate post-MI remodeling. However, it is unclear whether there is a synergistic effect by modulating both signaling pathways for heart repair. Furthermore, the majority of drugs are administrated to patients by simple systemic delivery, which generally leads to adverse off-target effects, drug toxicity, and low treatment efficacy. In addition, a holistic approach is still required to regenerate damaged human heart by targeting multiple tissue pathologies, including remuscularization, electromechanical stability, angiogenesis, resolution of fibrosis, and immunological balance. Therefore, a biomaterial-based therapeutic microporous hydrogel scaffold 10 with localized multi-drug delivery may be necessary to promote cardiac regeneration by providing pleiotropic pharmaceutic effects.

To this end, the injectable, multi-modal therapeutic microporous hydrogel scaffold 10 was developed for MI therapy. The generation of a therapeutic microporous hydrogel scaffold 10 is shown schematically in FIGS. 1A-1C. Hydrophobic therapeutic agents or drugs 16 were loaded into nanoparticles 14 (NPs), which were further encapsulated into matrix metalloprotease (MMP) sensitive PEG-based spherical µGel particles 12 or beads to generate the building blocks, i.e., drug/NPs-µGel beads, using a flow-focusing microfluidic device 200 as seen in FIGS. 1B, 2A, 2B. When the collection of spherical hydrogel particles 12 or building blocks were injected into the infarcted heart tissue 100, endogenous factor XIIIa (FXIIIa) could activate peptide K (Pep-K) and peptide Q (Pep-Q) present in/on the particles 12 to induce surface binding between the particles 12 or µGel beads and form contiguous therapeutic microporous hydrogel scaffold 10 in situ. By co-loading hydrophobic drugs 16 of F and R, the injectable hydrogel 12 pre-annealed slurry of particles 12 then forms the therapeutic microporous hydrogel scaffold 10 in situ and endow pleiotropic benefits for heart repair by providing mechanical support, promoting cell migration and neovascularization, while suppressing fibrosis and immune responses.

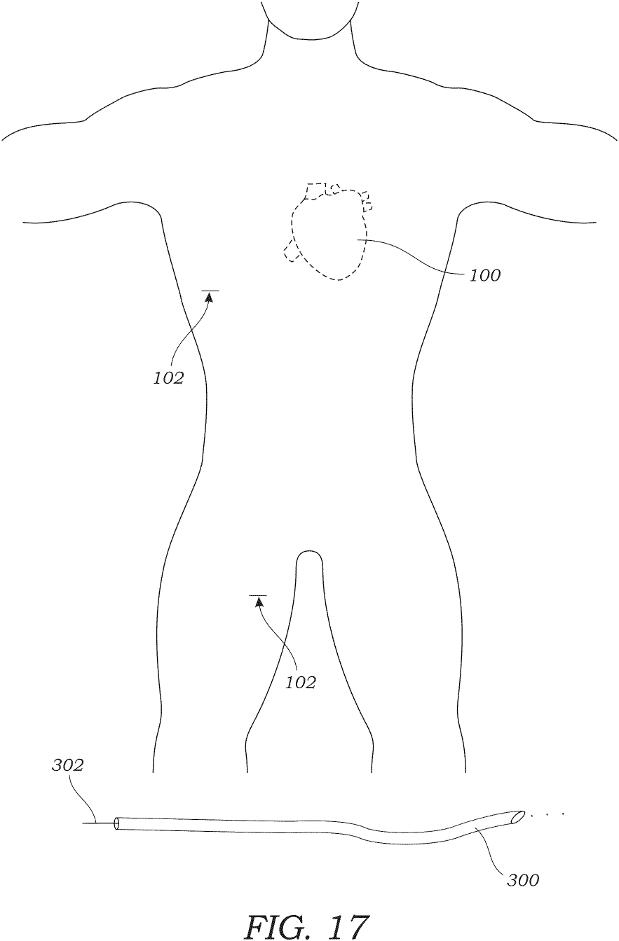

The pre-annealed slurry of particles 12 that forms the therapeutic microporous hydrogel scaffold 10 may be injected directly into the heart tissue 100 using a delivery device 300 as illustrated in FIG. 17. For example, a catheter-like device 300 may be used to inject the unannealed hydrogel slurry of particles 12 to an internal surface of the heart 100 (via the internal chambers of the heart) that are then annealed in situ as explained herein. Alternatively, the external tissues of the heart 100 may be injected with the pre-annealed hydrogel slurry using an endoscopic or laparoscopic device 300. A small incision in the chest or other access points 102 may be used to insert the delivery device 300 (FIG. 17). In one embodiment, the spherical hydrogel particles 12 may anneal to one another in response to an endogenous annealing agent (e.g., factor XIIIa (FXIIIa)). Alternatively, the spherical hydrogel particles 12 may anneal in response to an exogenous annealing agent (e.g., also FXIIIa). This exogenous annealing agent may be delivered by the same or different delivery device 300 that delivers the hydrogel pre-annealed slurry of particles 12. The distal end 302 of the delivery device 300 may be pre-loaded with the hydrogel particle 12 slurry and optional annealing agents which can be ejected out of the needle 302 and into the heart tissue 100. The tissue 100 that may be treated may include diseased tissue (e.g., the infarct region or border region of the infarct), healthy heart tissue, and combinations of the same. In yet another alternative, the spherical hydrogel particles 12 may anneal through a combination of both an endogenous annealing agent and an exogenous annealing agent.

While the therapeutic microporous hydrogel scaffold 10 that is disclosed herein has particular applicability to the treatment of MI, the therapeutic microporous hydrogel scaffold 10 may be used in a wide range of tissue types and biomedical applications. This includes tissue repair, tissue regeneration (heart, brain, skin, nerve, skeletal muscle, liver, lung, kidney, bone, etc.), cancer therapy, and immune modulation (e.g., vaccines). Of course, different therapeutic agents or drugs 16 would be loaded into the nanoparticles 14, depending on the ultimate application.

### Experimental

### Results and Discussion

### Development of Hydrogel Scaffold Building Blocks

The microfluidic device 200 for the generation of the spherical hydrogel particles 12 used in the therapeutic microporous hydrogel scaffold 10 was designed and fabricated with soft lithography (FIGS. 1B and 2A). The microfluidic device 200 has a first aqueous phase microfluidic channel 202a and a second aqueous phase microfluidic channel 202b interfacing in an emulsion generating region 204, the emulsion generating region 204 interfacing with a first oil phase microfluidic channel 206a and a second oil phase microfluidic channel 206b. As illustrated in FIG. 2A, additional oil phase microfluidic channels 208a, 208b may be provided to add oil along with a higher concentration of surfactant to prevent downstream coalescence of the spherical hydrogel particles 12. To make the spherical hydrogel particles 12, a first aqueous solution is flowed into the first aqueous phase microfluidic channel 202a via inlet 203a that includes PEG vinyl sulfone pre-reacted with K-peptide, Q-peptide, RGD peptide (optional), and a plurality of nanoparticles 14 loaded with one or more therapeutic agents or drugs 16. A second aqueous solution is flowed into the second aqueous phase microfluidic channel 202b via inlet 203b that includes a MMP-sensitive crosslinker. Oil and a surfactant (e.g., Pico-Surf™ or Span) is flowed into the first and second oil phase microfluidic channels 206a, 206b. Oil and a higher concentration of surfactant (as compared to what is present in microfluidic channels 206a, 206b) is flows through microfluidic channels 208a, 208b via inlets 209a, 209b to generate emulsions of the mixed first and second aqueous solutions in the oil at the emulsion generation region 204. The now-formed emulsions enter the droplet collection region 210 are then allowed to crosslink to form spherical hydrogel particles 12. An outlet 212 is provided to remove the spherical hydrogel particles 12.

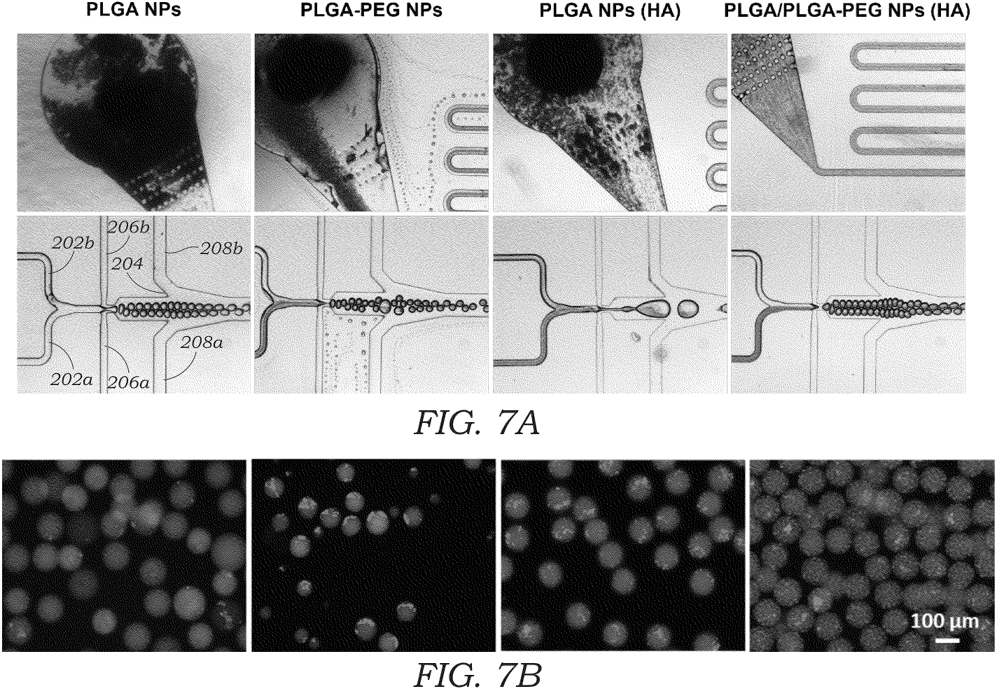

To achieve sustained drug release from the therapeutic microporous hydrogel scaffold 10, biodegradable poly(lactic-co-glycolic acid) (PLGA) based polymers were used to make therapeutic agent/drug-containing nanoparticles 14 by an emulsification solvent evaporation technique, and mixed with the pre-gel solutions prior to µGels 12 formation. PLGA is a biodegradable polymer being used in many FDA-approved products, and PLGA-based particles have been widely employed for drug delivery because of their biocompatibility and controllable biodegradation. However, the hydrophobic PLGA nanoparticles 14 aggregated and precipitated quickly in the aqueous pre-gel solution, leading to failure in the production of NPs-µGels in the microfluidic device 200, because of blockage or leakage of the microfluidic channels 202, and unstable processing which caused the generation of heterogenous low-quality µGels (FIGS. 7A and 7B).

In response, two strategies were employed to suspend the PLGA NPs 14 and delay particle aggregation in the pre-gel solution through improving NP 14 surface hydrophilicity and increasing the viscosity of the pre-gel solution (FIGS. 7A and 7B). The mean hydrodynamic diameter of NPs 14 was ~400 nm with a polydispersity index of 0.23 as measured by dynamic light scattering (DLS). The NP surface hydrophilicity was adjusted in the aqueous pre-gel solutions by using different PLGA-PEG copolymers, including PLGA 35k, PLGA55k-b-PEG5k, and PLGA25k-b-PEG5k. It was found that particle suspension was enhanced with the increase of PEG length, and PLGA55k-b-PEG5k NPs 14 resulted in a stable preparation of NPs-µGel. However, a further increase of hydrophilicity might lead to a lower encapsulation capacity of hydrophobic drugs and cause a burst drug release. Thus, another strategy was implemented in addition to the adjustment of the particle hydrophilicity. Here, through the addition of hyaluronic acid (HA) to the pre-gel solution, the solution viscosity was increased, resulting in a reduction of NPs 14 aggregation. After optimization, it was found that the NPs 14 made by PLGA35k/PLGA55k-PEG5k (1:1 weight ratio or substantially equivalent) and the addition of 0.25 v/v% HA in pre-gel solution achieved a stable preparation of NPs-µGel particles 12 with uniform size, controlled NPs loading, and uniform NPs distribution (FIGS. 2C-2E). In some embodiments, the amount of HA in pre-gel solution is less than about 1% v/v% hyaluronic acid (HA). To monitor and visualize the mixing process of the two aqueous phases and the particle distribution in the spherical hydrogel particles 12, NPs 14 were labeled with coumarin-6 and the pre-gel solution was conjugated with AF546-maleimide (FIG. 2F). The fluorescent images show that NPs 14 were uniformly encapsulated in the spherical hydrogel particles 12 or µGel beads (FIG. 2G). Thus, PLGA35k/PLGA55k-PEG5k NPs 14 were used as the drug loading material for all the subsequent studies.

### Characterization of Building Blocks and Scaffolds Formed Thereby

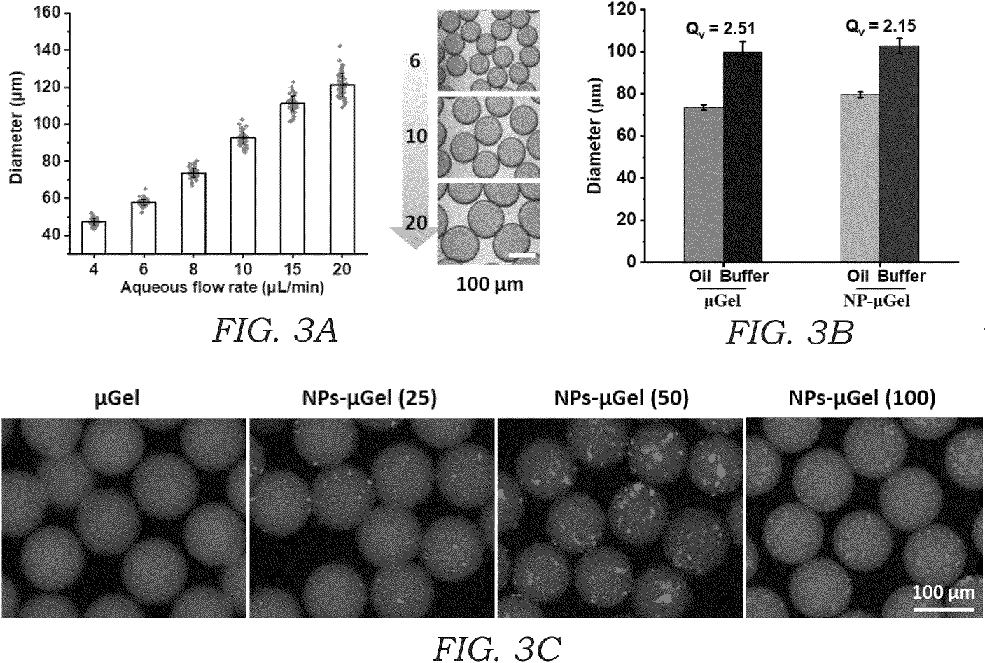

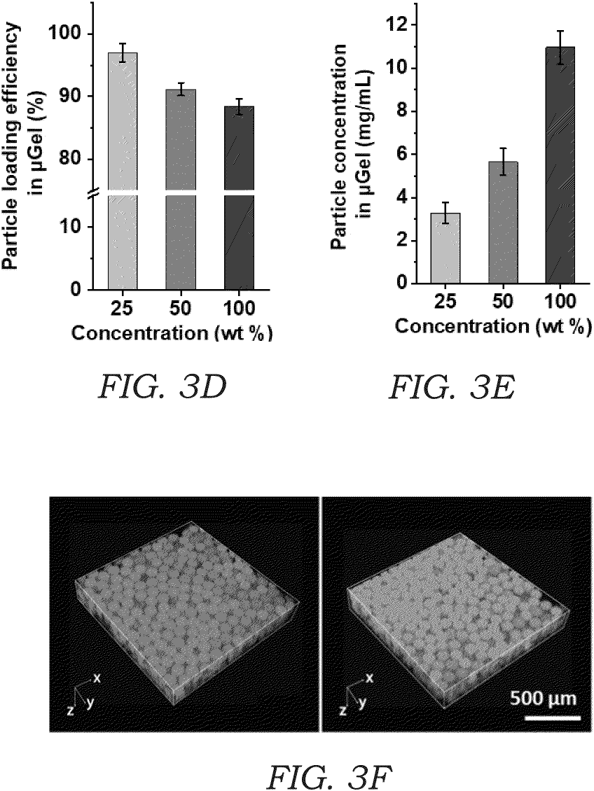

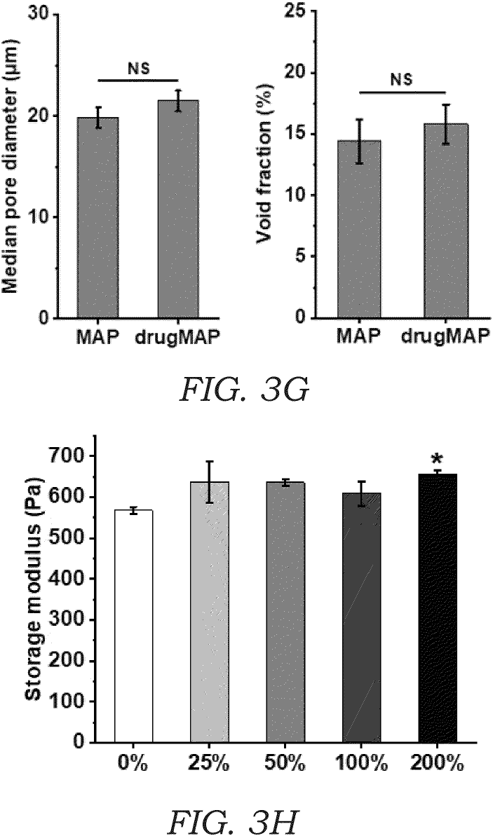

A major advantage of the microfluidic-emulsion technique is the production of highly monodisperse hydrogel microparticles 12 of well-defined size. NPs-µGel particles 12 or beads could be produced with diameters ranging from 45 µm to 120 µm by tuning the flow rate of aqueous solutions into the microfluidic device 200 (FIG. 3A). Some minor differences in particle 12 formation were observed for particles 12 containing NPs 14. In particular, at an aqueous flow rate of 8 µL/minutes, NPs-µGel particles 12 or beads in the oil phase were larger than particles 12 without NPs 14, potentially because the addition of HA and NPs 14 increased the viscosity of aqueous solution and affected the droplet breakup. However, NPs 14 encapsulation slightly decreased the gel swelling ratio in buffer solution, resulting in the final diameter of NPs-µGel particles 12 still being similar to µGel particles (~100 µm) (FIG. 3B).

To adjust the drug delivery capacity of the therapeutic microporous hydrogel scaffold 10, gel droplets loaded with different amounts of NPs 14 from 0% to 100% (weight of NPs/weight of dry pre-gel components) were prepared (FIG. 3C). After gelling and purification, the particle loading efficiency was higher than 90% for all tested NPs-µGel particles 12 (FIG. 3D), and the final NP concentration in the gels was highly correlated with the initial loading amount (FIG. 3E). The NPs 14 that were not encapsulated in the particles 12 might be lost in the device 200 during gel fabrication or released during gel purification. The therapeutic microporous hydrogel scaffold 10 generated from 100 µm diameter NPs-µGel particles 12 maintained an interconnected porous structure after annealing (FIG. 3F) with a median pore diameter ~ 20 µm and ~15% average void fraction (FIG. 3G). With pores of these dimensions, cells can easily infiltrate and traverse the therapeutic microporous hydrogel scaffold 10 even before degradation of the scaffold. In addition, the pore diameters could be adjusted by tuning the particle 12 or building-block sizes. The loading of NPs 14 did not affect the ability of NPs-µGel particles 12 to anneal to form contiguous microporous hydrogel scaffolds 10. In vitro, the particles 12 were annealed via activated FXIIIa, in which a non-canonical amide covalent bond formed between the ε-amine of lysine in peptide-K and the γ-carboxamide of glutamine in peptide-Q on the microbeads. When the particles 12 were injected in vivo, the endogenous thrombin and FXIIIa could induce the crosslinking of the particles 12 to form the therapeutic microporous hydrogel scaffold 10 in the infarcted heart. Mechanical properties are critical biophysical cues in MI therapy. Therefore, the influence of nanoparticle 14 loading on the mechanical stiffness of hydrogel was investigated. The results demonstrated that the addition of NPs 14 in gels had a negligible effect on the hydrogel stiffness, yielding a storage modulus of ~600 Pa (FIG. 3H). The stiffness is in the same order as the stiffness of other soft hydrogels, which have shown improved therapeutic outcomes in post-MI therapy. In particular, the therapeutic microporous hydrogel scaffold 10 provides a porous structure for fast cell infiltration and mechanical support immediately after injection, and the mechanical properties of the therapeutic microporous hydrogel scaffold 10 can be easily adjusted to achieve stiffness matching between the scaffold 10 and native tissue via modulating the stiffness of individual µGel particles 12, annealing chemistry, crosslinking degree and particle-packing density.

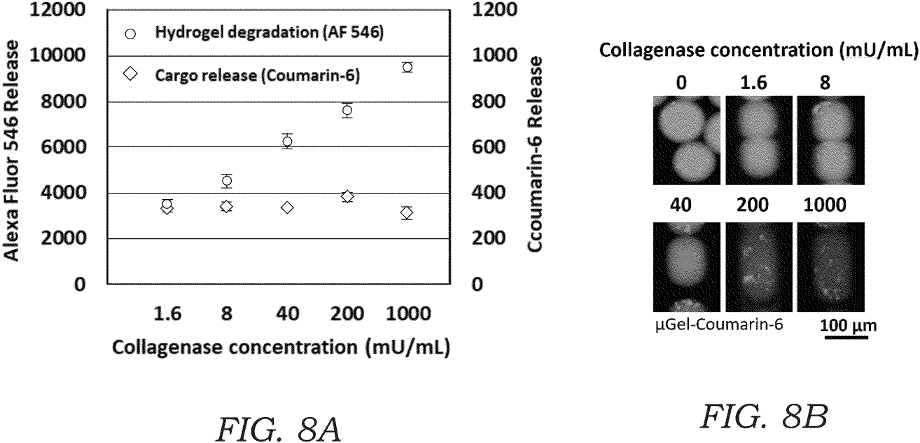

The degradation of biomaterials enables increased in situ tissue regeneration as the material is replaced by cells and ECM. The therapeutic microporous hydrogel scaffold 10 gel mesh was crosslinked with MMP-sensitive peptide, making it degradable by MMP enzyme. MMPs are highly relevant to cardiac remodeling after MI as the MMP9 level is elevated in plasma and left ventricle after MI in animals and humans. To check the therapeutic microporous hydrogel scaffold 10 gel degradation in enzyme solution and address whether gel degradation affected the drug release profile, Coumarin-6 was loaded into NPs-µGel particles 12 as a hydrophobic fluorescent model drug and characterized the degradation of pelleted NPs-µGel particles 12 in the presence of MMP enzyme (collagenase II) in vitro (FIG. 8A). It was found that the NPs-µGel particles 12 degraded faster with the increase of collagenase concentration. However, the release of coumarin-6 in NPs 14 was not affected by changing the concentrations of collagenase. Fluorescence imaging of NPs-µGel particles 12 showed a direct correlation between the collagenase concentration and the extent of degradation (represented by diminishing AF546 signal intensity) as well as particle deformability (evidenced by elongation and swelling of the particles) (FIG. 8B). Furthermore, it was found that NPs 14 also increased in size during degradation and remained trapped inside µGel particles 12. There might be two possible reasons for the particle trapping in the hydrogel 10 mesh during degradation. First, the ester bonds of polyester could be hydrolyzed to form hydrophilic carboxyl and hydroxyl groups, so the hydrophilicity of the particles 12 would increase gradually to promote water absorption, thus forming larger swollen particles 12 or clusters. Secondly, the carboxyl groups of polyester fragments could interact with the amine groups of gel components electrostatically. Overall, these in vitro data suggested that the drug release profile from the therapeutic microporous hydrogel scaffold 10 remained relatively independent of gel degradation.

### In Vitro Evaluation of Drugs and Microporous Hydrogel

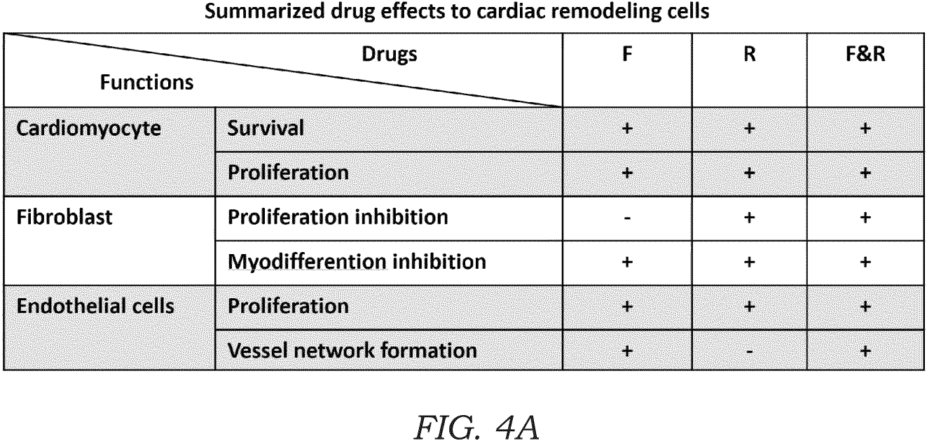

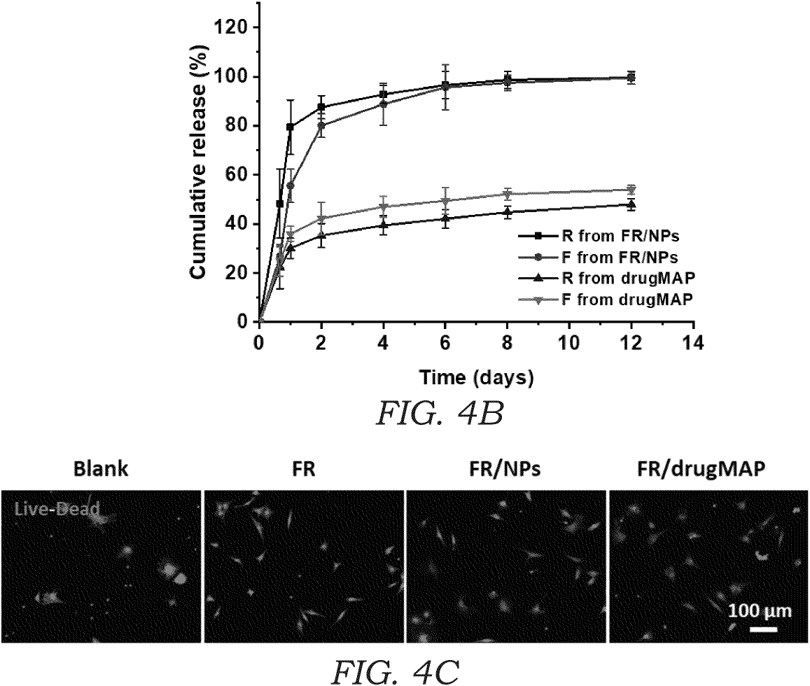

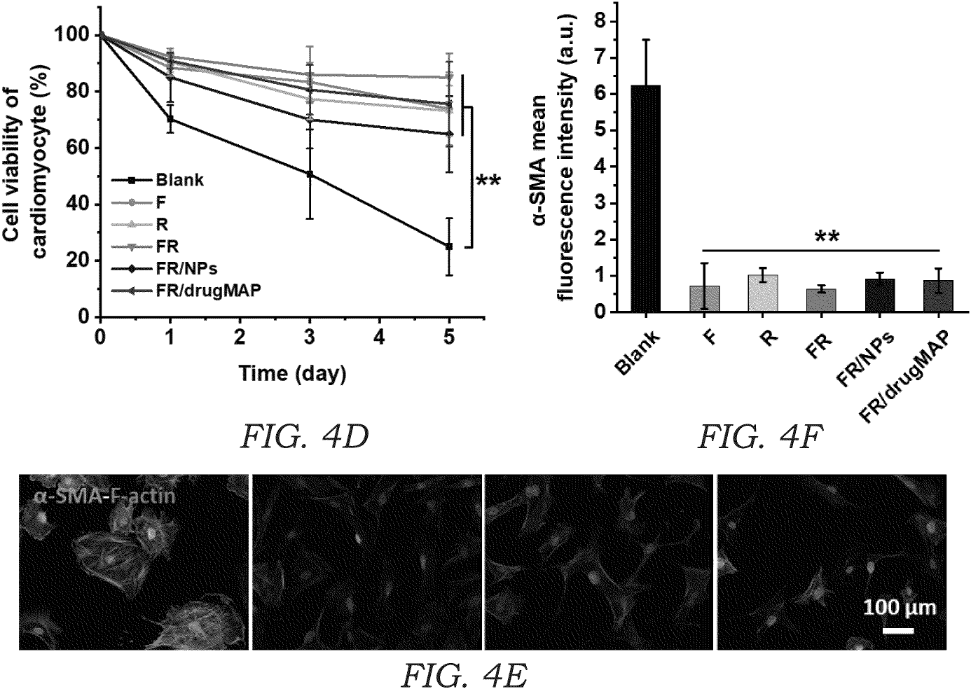

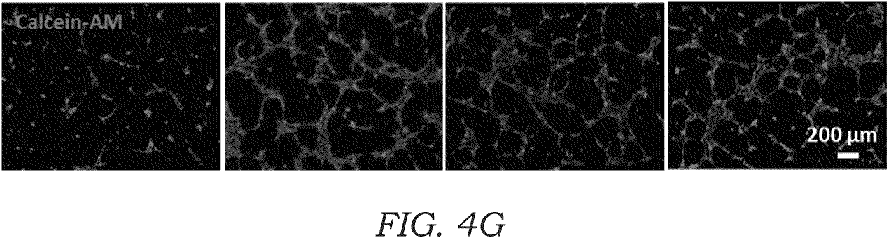

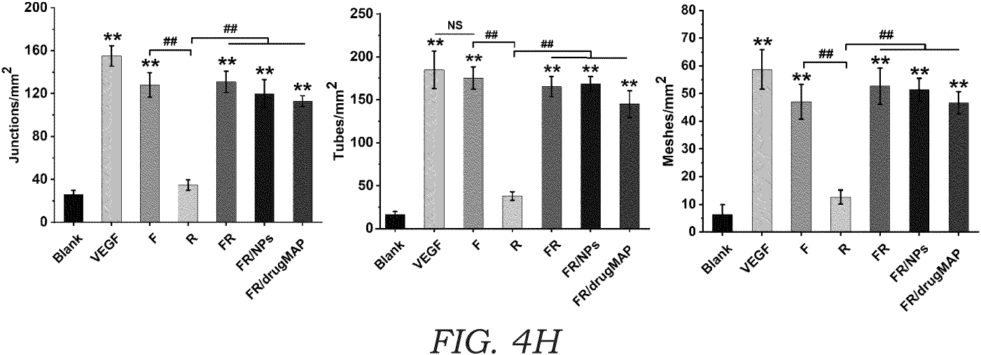

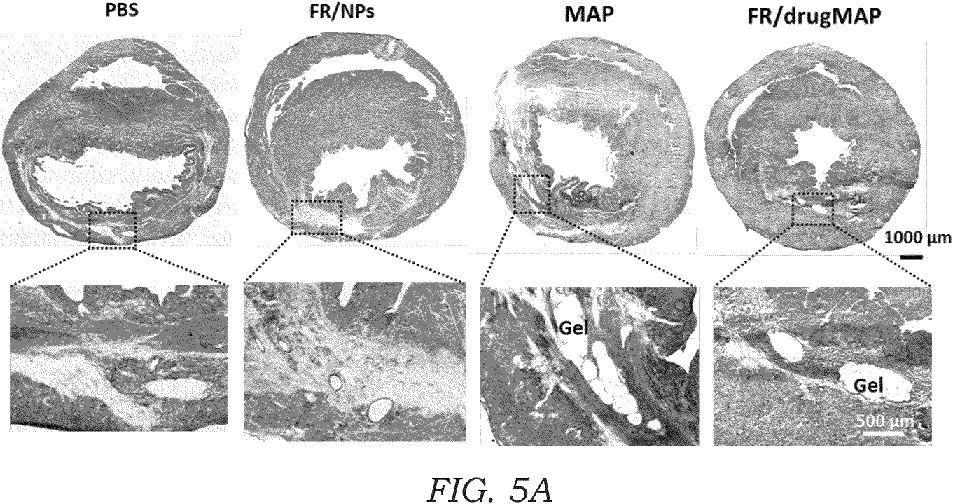

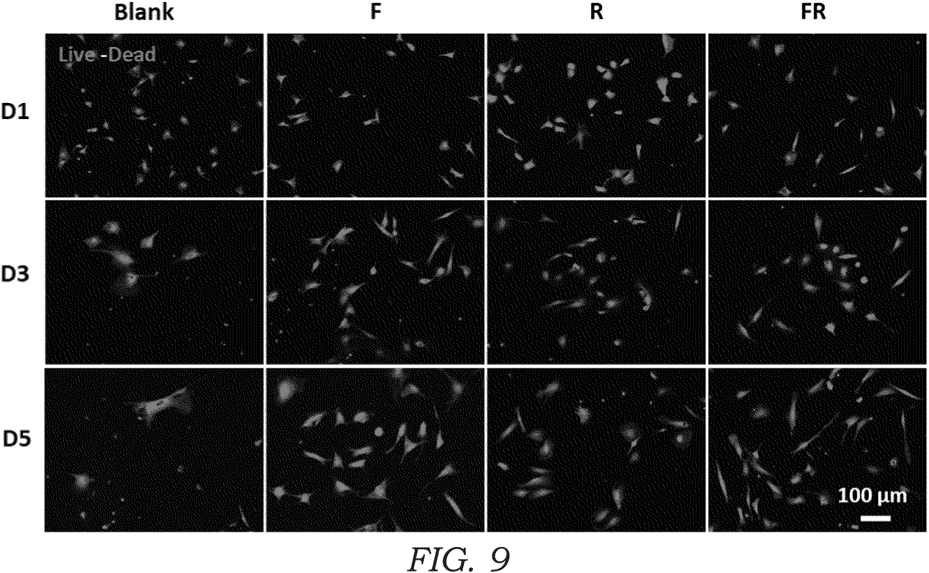

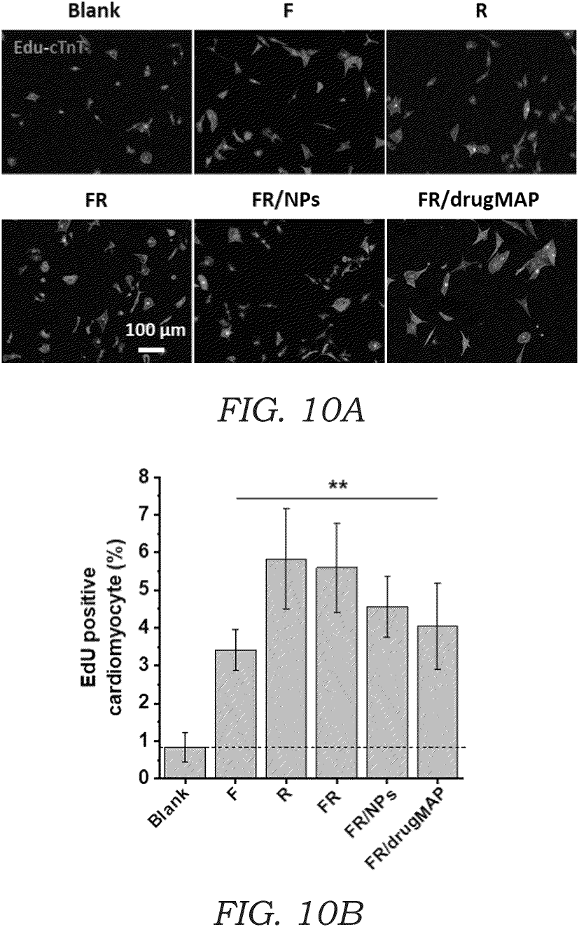

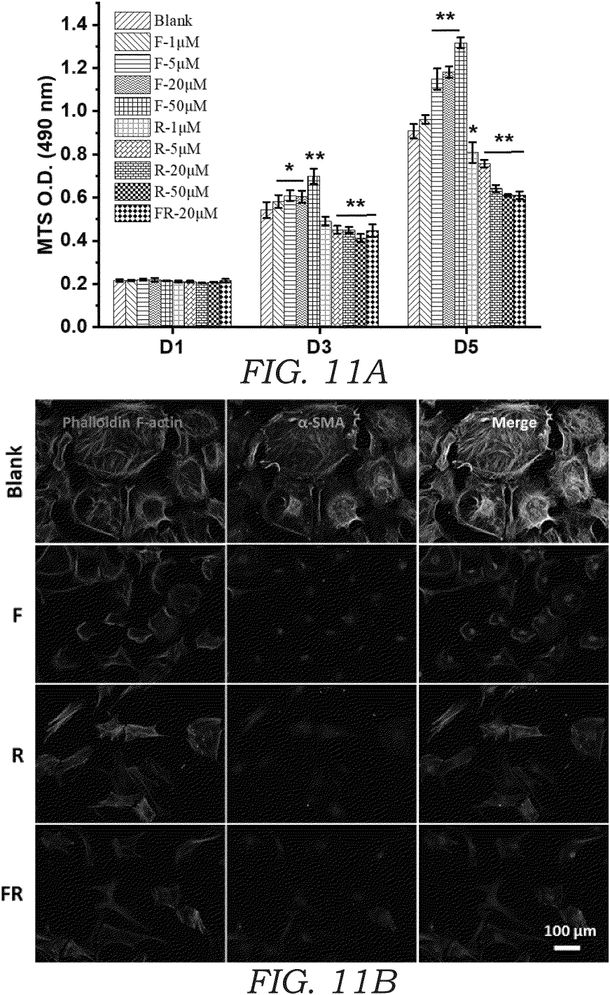

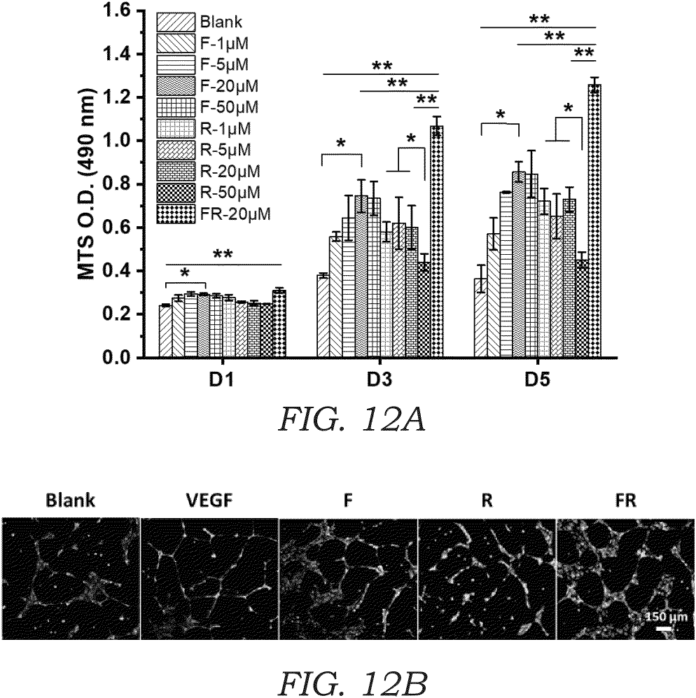

Previous studies have reported that F and R have specific effects on preventing cardiac dysfunction, respectively. However, their effects on various cell types in cardiac tissues have not been systematically evaluated, and it is not clear whether the combination of F and R has additive or synergistic effects. In the initial drug evaluation, it was found that both F and R or FR combination could maintain cardiomyocyte viability at 80 % after 5 days in vitro culture, which was significantly higher than 25 % for control cells (FIG. 9). In addition, both F and R significantly enhanced the proliferation of neonatal cardiomyocytes, yielding three and six times as many cells as the control, respectively (FIGS. 10A-10B). For cardiac fibroblasts (FIG. 11A), F showed dose effects to enhance fibroblast proliferation, in contrast, R showed the opposite inhibitory effects. Nevertheless, the inhibition of fibroblast proliferation can be maintained when both drugs used together. It was also found that each F or R, or their combination could prevent myo-differentiation of cardiac fibroblasts (FIG. 11B). Furthermore, for endothelial cell (EC) proliferation and tubule network formation (FIGS. 12A-12B), both F and R showed dose-dependent effects to enhance EC proliferation, and their optimal concentration was the same (20 µM). Notably, EC proliferation was significantly enhanced with the combination of the two drugs. EC network formation was increased with F or FR treatment, while not with R alone. Altogether, the collected effects of both drugs on cardiac cells were summarized in FIG. 4A. Since F and R had additive and complementary benefits in promoting cardiomyocyte survival, inhibiting fibroblast myo-differentiation and enhancing EC proliferation and tubule formation, both hydrophobic agents were loaded into PLGA-based NPs 14 (FR/NPs), which were further encapsulated into µGel particles 12 to generate FR/particle 12 building blocks.

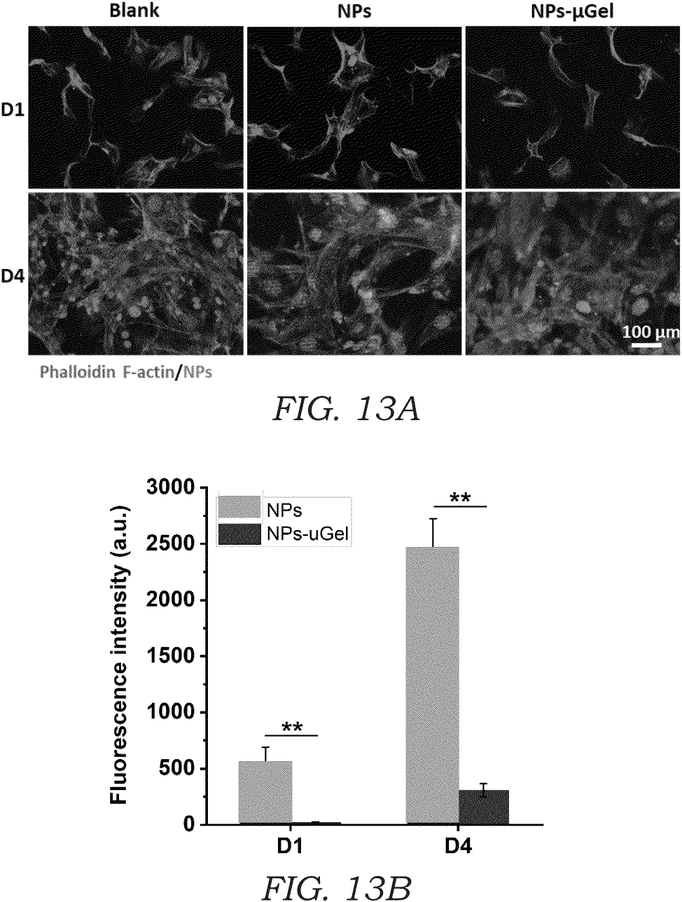

The drug release profiles from FR/therapeutic microporous hydrogel scaffold 10 demonstrated that both hydrophobic chemicals were gradually and simultaneously released throughout 2 weeks (FIG. 4B). It was found that less F and R were released from the therapeutic microporous hydrogel scaffold 10 versus NPs 14 alone, which might be explained by a burst release from NPs 14 which already occurs during the production phase of the spherical hydrogel particles 12. The in vitro biological evaluations were further performed for the drug-releasing platforms (FIGS. 4C-4F). Similar to FR added directly to the medium, FR/NPs 14 and FR/therapeutic microporous hydrogel scaffold 10 yielded the combined beneficial effects and significantly enhanced cardiomyocyte survival compared to control (FR/NPs: 65%, FR/therapeutic microporous hydrogel scaffold: 75% vs. blank: 25% at day 5) (FIGS. 4C-4D). In addition, both alpha-smooth muscle actin (α-SMA) and F-actin were strongly expressed in the blank control (FIGS. 4E-4F), and there were no differences between the blank control and the supernatants from the unloaded NPs or blank µGels (FIGS. 11A-11B). However, same as adding drugs (F and R) in the medium, both FR/NPs and FR/therapeutic microporous hydrogel scaffold diminished fibroblast myo-differentiation with significantly lower α-SMA expression. In parallel, there was a decrease of F-actin in response to released F and R, suggesting that the formation of actin stress fibers was blunted in parallel with the decrease in α-SMA expression, consistent with a previous finding. Moreover, both FR/NPs and FR/therapeutic microporous hydrogel scaffold obviously enhanced EC vascular network formation (FIG. 4G), and exhibited significant higher number of junctions, tubes and meshes versus the blank control (FIG. 4H). Taken together, these in vitro results demonstrated the beneficial effects of FR/therapeutic microporous hydrogel scaffold on regulating cardiac remodeling cells, including enhancing cardiomyocyte survival, inhibiting fibroblast myo-differentiation and promoting endothelial cell proliferation and tubule formation. Beyond the controlled drug release, the cellular uptake of NPs 14 embedded in µGel particles 12 was found to be significantly reduced, compared to free NPs (FIGS. 13A-13B), which could decrease the cytotoxicity and inflammatory response.

### Cardiac Function Improvement With Injection of NPs-µGel

To investigate the potency for cardiac repair with the therapeutic microporous hydrogel scaffold, rat MI models were created by ischemia-reperfusion injury through the ligation of the left anterior descending artery. As previous studies suggested that the best therapeutic outcomes of hydrogel-based approaches were found 2-3 days after MI, injections were performed 2 days after infarction of four randomized groups with the treatments of PBS (n = 9), FR/NPs (n = 6), MAP gel (n = 9) and FR/NPs-µGel used to form microporous hydrogel scaffold 10 (n = 9), respectively.

Solutions were successfully injected into the infarcted zone by ultrasound-guided transthoracic injection (total 100 µL with two sites of 50 µL injections). The commonly used natural and synthetic hydrogels usually undergo a solution-to-gel transition upon stimulus exposure, while it is relatively difficult to control and balance the ideal solution-to-gel transition time. Inappropriate gelation speed may lead to many adverse effects. Slow gelation (from minutes to hours) could increase tissue necrosis or the loss of materials and therapeutic molecules. On the other hand, rapid gelation (from seconds to minutes) leads to quick needle blockage, handling inconveniences and limited tissue integration. Unlike commonly applied bulk hydrogels, the particle 12 gel building blocks are flowable as a slurry and can be easily injected into highly motile cardiac tissue and stay at the injection site without gel dislodgment, which might avoid the handling issues and risks of rapid or slow gelation.

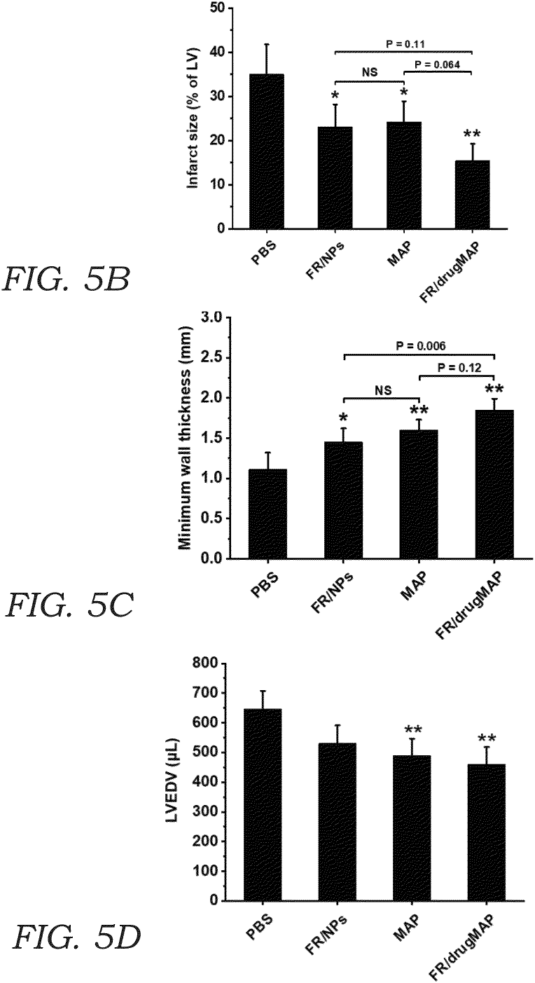

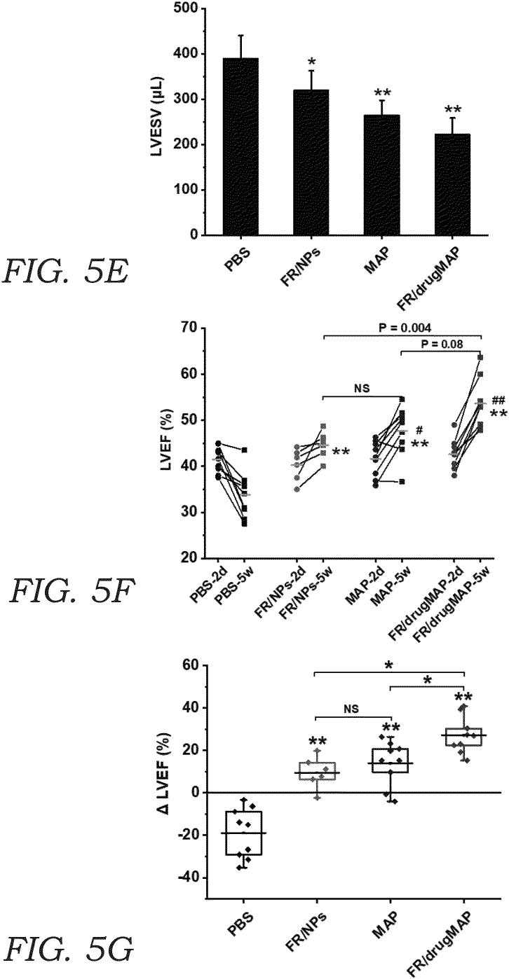

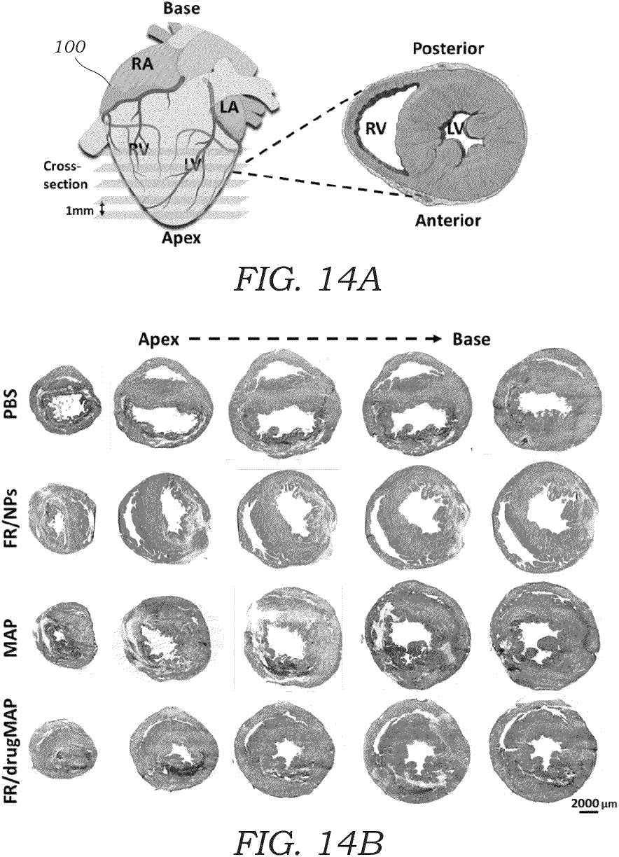

The MI therapeutic outcomes of all groups were evaluated at 5 weeks posttreatment by histology and echocardiography analysis. Masson’s trichrome staining showed the gross heart morphology and revealed less MI region, fibrosis, LV dilation and wallthinning in hearts treated with FR/NPs- or MAP gel-only groups compared with PBS group, with further improvement for hearts treated with integrated FR/therapeutic microporous hydrogel scaffold 10 (FIG. 5A, FIGS. 14A-14B), resulting in the smallest infarct size (FR/therapeutic microporous hydrogel scaffold 10: 15.4 ± 3.9% vs. PBS: 35 ± 6.8%; FR/NPs: 23.1 ± 5.1%; MAP: 24.2 ± 4.7%; FIG. 5B) and the thickest minimum LV wall (FR/therapeutic microporous hydrogel scaffold 10: 1.85 ± 0.14 mm vs. PBS: 1.11 ± 0.21 mm; FR/NPs: 1.45 ± 0.17 mm; MAP: 1.6 ± 0.13 mm; FIG. 5C). In addition, the reduced cardiac remodeling of FR/NPs, MAP gel and FR/ microporous hydrogel scaffold-treated groups was further demonstrated by the reduction in left ventricle end-diastolic volume (LVEDV) and end-systolic volume (LVESV), respectively, compared with PBS controls (FIGS. 5D and 5E). The ventricular ejection fractions (LVEF) at day 2 baseline were similar between all groups, indicating a similar degree of initial MI injury (FIG. 5F). However, after 5 weeks, the LVEF of PBS-treated group distinctly declined, while LVEF was well preserved in the FR/NPs, MAP gel and FR/therapeutic microporous hydrogel scaffold-treated groups. Notably, FR/therapeutic microporous hydrogel scaffold-treated rats displayed the best LV contractility of infarcted hearts with the highest LVEF (FR/ microporous hydrogel scaffold: 53.6 ± 5.2% vs. PBS: 33.7 ± 4.9 %; FR/NPs: 44.9 ± 3.1%; MAP: 47.7 ± 5.3%; FIG. 5F) and the highest therapeutic efficiencies (change of LVEFs from baseline, FIG. 5G). Overall, the cardiac remodeling was significantly attenuated by the treatment with FR/NPs or MAP gel alone, indicating the respective benefits of the drugs (F and R) and hydrogel-based mechanical support in ameliorating post-MI remodeling and rescuing cardiac dysfunction. Compared to treatment alone, the integrated FR/therapeutic microporous hydrogel scaffold 10 showed the best therapeutic outcomes.

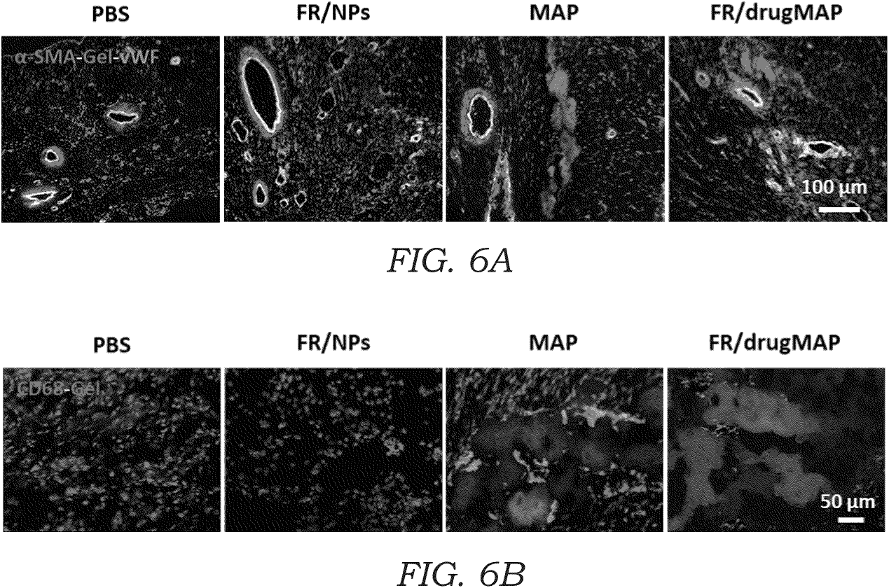

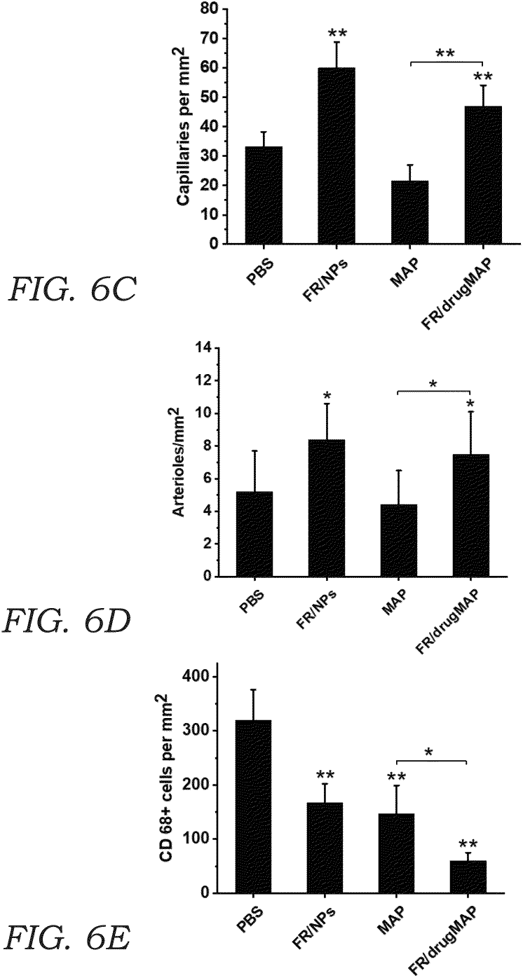

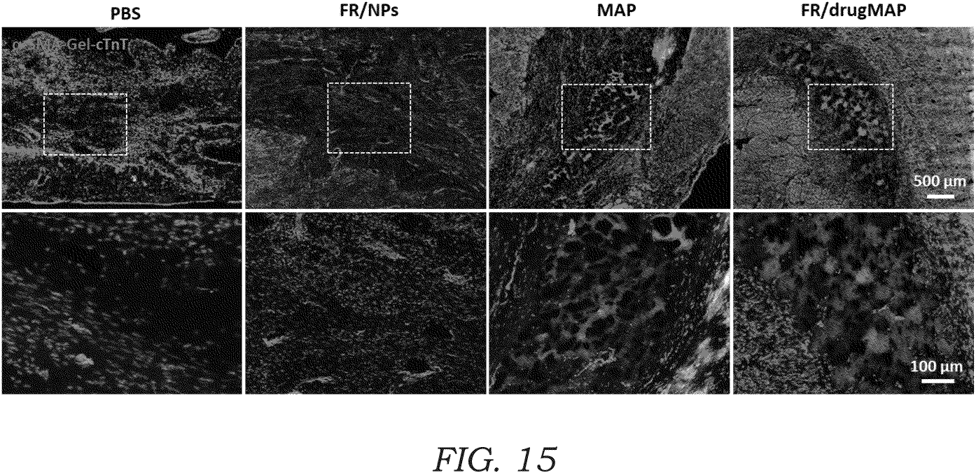

To reveal the underlying mechanisms for the functional effects of the therapeutic microporous hydrogel scaffold 10, immunostaining analysis was further performed and assessed angiogenesis and immune response in the infarcted hearts (FIG. 6). Infarcted hearts were stained with von Willebrand factor (vWF, for ECs) and α-SMA (for smooth muscle cells) (FIG. 6A, FIG. 15), and the results showed that the numbers of both capillaries (vWF⁺) and arterioles (α-SMA⁺) were significantly increased in FR/NPs-treated and

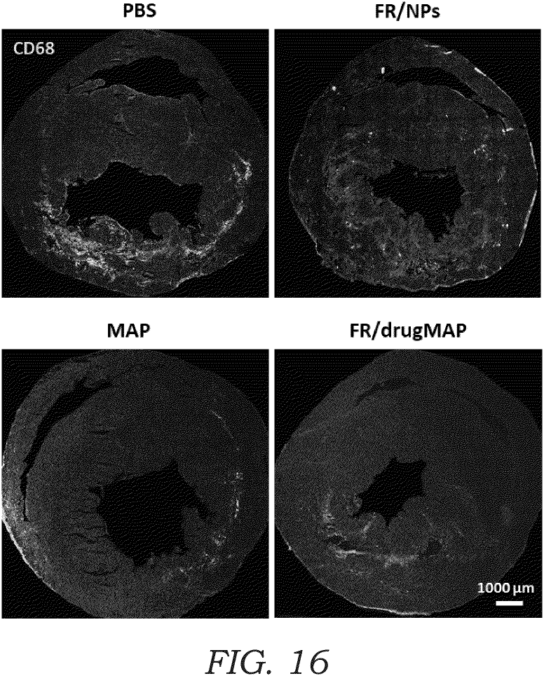

FR/therapeutic microporous hydrogel scaffold-treated groups in comparison to PBS and MAP-treated groups (FIGS. 6C and 6D). Notably, the FR/ microporous hydrogel scaffold-treated hearts exhibited prominent angiogenesis, while there was less angiogenesis treated with MAP gel alone, suggesting that the drugs further promote neovascularization. Additionally, in contrast to PBS-treated hearts, other three treatments showed less CD68⁺ macrophage infiltration in the infarcted hearts, especially for the FR/therapeutic microporous hydrogel scaffold-treated group (FIG. 6B, FIG. 6E, FIG. 16), demonstrating that both drug and MAP gel could reduce the inflammatory responses in MI hearts, and their combination and integration could further enhance the efficiency. Together, these in vivo results suggest that the integrated therapeutic microporous hydrogel scaffold 10 could enhance the MI therapeutic effects through the promotion of neovascularization and the inhibition of inflammatory response.

To date, numerous injectable hydrogels have been investigated for cardiac repair and regeneration. However, rapid host tissue integration and spatiotemporal control of biologics presentation are challenges for most natural and synthetic bulk hydrogels, which can compromise the efficacy of the hydrogel-based therapy for cardiac repair. In recent years, very few granular hydrogels have been exploited in tissue repair. By annealing the µGel particles 12 to form porous scaffolds 10, the granular hydrogel permits several noteworthy features. First, the small size of µGel particles 12 enables minimally invasive injection. Secondly, the modular particles 12 or building blocks makes it flexible to engineer multiscale physical properties by varying polymer composition, µGel shape, size and stiffness, and interparticle friction. Thirdly, granular hydrogels possess porosity and diffusivity and can be tuned to support cell proliferation and migration. For example, the injection of granular porous hyaluronic acid hydrogels into myocardial tissues demonstrated the degradation behavior and cell invasion after 3 weeks. However, the MI therapeutic outcomes were not evaluated by histology and echocardiography analysis.

Injectable hydrogels are promising for localized drug and cell delivery in many biomedical applications. Current granular hydrogel systems have been used for the sustained delivery of hydrophilic biologics (cells and drugs). For example, heparin has been incorporated into microparticles to sustain the delivery of growth factors through electrostatic associations. Similarly, protein activators or inhibitors such as antibodies can also be delivered, while they are more expensive and may lose activity through proteolytic enzymatic digestion and degradation over time. In contrast, small molecules are generally more stable, cheaper, and easier to be loaded into a drug delivery system. However, it is still challenging to pack hydrophobic drugs into microfluidic-generated granular hydrogel systems. Delivery of hydrophobic drugs or cargos can be controlled by loading the drugs into hydrophobic carriers (such as NPs 14). However, these hydrophobic particles can aggregate into clusters and precipitate quickly in the hydrophilic pre-gel solution, resulting in the blockage of microfluidic channels and unstable drug loading, as shown herein. Unstable loading into µGel particles 12 leads to loss of particles 12, and inconsistent drug dosing. Here, uniform encapsulation of hydrophobic drug-loaded NPs 14 within microfluidic-generated hydrophilic µGel particles 12 was accomplished by modulating NP surface hydrophilicity and the viscosity of the pre-gel solution for controlled hydrophobic drug delivery.

In the experiments disclosed herein, F and R were evaluated and loaded into the therapeutic microporous hydrogel scaffold 10 for MI therapy. Both hydrophobic drugs 16 can be sustained release in two weeks in vitro. There was a partial release of both drugs 16 during the production phase of therapeutic microporous hydrogel scaffold 10, due to the burst release occurring when the NPs 14 were suspended in an aqueous pre-gel solution or embedded in gel. Depending on the therapeutic purpose, the drug release period from NPs 14 can be tailored from hours to months, by tuning the polymer composition, molecular weight, and the content of the hydrophilic block. Besides the intrinsic release profile from drug-loaded NPs 14, the amount of NPs 14 encapsulated in each µGel particle 12 and the volume of µGel particles 12 are also critical parameters to determine the overall drug release profile and pharmacologic effects. Furthermore, the MI therapeutic outcomes of therapeutic microporous hydrogel scaffold 10 systems by histology, echocardiography and immunostaining were systematically analyzed. It was found that the integrated FR/therapeutic microporous hydrogel scaffold could significantly ameliorate cardiac remodeling and dysfunction, in comparison to FR/NPs-only and MAP-only groups, by inhibiting fibrosis and inflammatory response, and promoting cell migration and neovascularization. It is worth noting that the therapeutic microporous hydrogel scaffold 10 has shown partial degradation in vivo after 5 weeks.

The therapeutic microporous hydrogel scaffold 10 overcomes challenges in integrating hydrophobic NPs 14 within microfluidic-generated hydrophilic µGel particles 12. The therapeutic microporous hydrogel scaffold 10 was loaded with hydrophobic drugs (F and R), and, prior to annealing, was injected into ischemic heart tissue 100, which promoted cardiac repair by offering multi-functional benefits, including fast cell infiltration, mechanical support, and synergistic pharmacological effects.

### Experimental Section

Microfluidic device fabrication: Droplet generating microfluidic devices 200 were fabricated by soft lithography as previously described in D. R. Griffin et al., Accelerated wound healing by injectable microporous gel scaffolds assembled from annealed building blocks, Nat. Mater. 2015, 14, 737, which is incorporated herein by reference. Briefly, master molds were fabricated on silicon wafers (University wafer) using two-layer photolithography with KMPR 1050 photoresist (Microchem Corp). The height for the droplet formation channel 202 was 50 µm, and the height for the collection channel 202 was 150 µm. Devices were molded from the masters using poly(dimethyl)siloxane (PDMS) (Sylgard 184 kit, Dow Corning). The base and crosslinker were mixed at a 10:1 mass ratio, poured over the mold and degassed before curing overnight at 65° C. Channels 202 were sealed by treating the PDMS mold and a glass microscope slide (VWR) with oxygen plasma (Plasma Cleaner, Harrick Plasma) at 500 mTorr and 80 W for 30 seconds. Thereafter, the channels 202 were functionalized by injecting 100 µL of Aquapel (88625-47100, Aquapel) and reacting for 30 seconds until washed by Novec 7500 (9802122937, 3 M). The channels 202 were dried by air suction and kept in the oven at 65° C. until used.

Preparation and characterization of drug-loaded NPs: An emulsification solvent evaporation technique was applied to prepare NPs 14. Briefly, different PLGA based polymers, including PLGA (Mw = 35 kDa, acid-terminated, cat# 26270, Polysciences), PLGA55k-b-PEG5k (PLGA average Mn = 55 kDa, PEG average Mn = 5 kDa, cat# 764752, Sigma), PLGA25k-b-PEG5k (PLGA average Mn = 25 kDa, PEG average Mn = 5 kDa, cat# 764752, Sigma), and mixed PLGA35k/PLGA55k-b-PEG5k (50/50 wt/wt) were dissolved in dichloromethane to make 10% w/v solutions. The resulting solution (1 mL) was added to stirred 3 mL 1% (w/v) PVA (Poly(vinyl alcohol), Mw = 25 kDa, 88% hydrolyzed, cat# 15132, Polysciences) solution using a vortex mixer at 2000 rpm for 2 minutes, and the emulsified polymer solution was immediately sonicated with a 20% amplitude (Sonic Dismembrator 500, Thermo Fisher Scientific) in six 10 second bursts. The test tube was immersed in ice water during sonication. After sonication, the emulsion was added dropwise into 30 mL 1% (w/v) PVA solution and stirred for 3 hours at room temperature to remove the residual organic solvent. NPs 14 were collected and washed three times with distilled water by centrifugation at 10,000 g for 5 minutes at 4° C., and the NPs were stored at -80° C. refrigerator. Particle diameter was measured by dynamic light scattering (DLS), and the surface morphology was observed by SEM with gold electrospray.

To prepare the fluorescence-labeled NPs 14, 0.02% (w/v) coumarin-6 (green fluorescence, Sigma) was added and dissolved in the polymer solution for nanoparticle fabrication. In addition, forskolin (cat# 11018, Cayman Chemical) and Repsox (cat# 14794, Cayman Chemical) were selected as the therapeutic agent or drug 16 and loaded into NPs 14 to generate FR/NPs. The aforementioned protocol was used, but 5% (wt/wt) of hydrophobic drugs with the same molar ratio of F and R were added and dissolved in the polymer solution of PLGA35k/PLGA55k-b-PEG5k (50/50 wt/wt).

Preparation of particle building blocks: The MMP sensitive PEG-based microgel (µGel) particles 12 used to form the therapeutic microporous hydrogel scaffold 10 were prepared by a customized microfluidic device 200 with two separate pre-gel aqueous solutions, as previously described, Griffin et al, supra. Aqueous solution 1: 10% (w/v) 4-arm PEG vinyl sulfone (Mw = 20 kDa, JenKem Technology USA Inc.) in 300 mM triethylamine (Sigma), pH 8.25, pre-reacted with 250 µM K-peptide (Ac-FKGGERCG-NH₂ [SEQ ID NO: 1], Genscript), 250 µM Q-peptide (Ac-NQEQVSPLGGERCG-NH₂ [SEQ ID NO: 2], Genscript) and 500 µM RGD peptide (Ac-RGDSPGERCG-NH₂ [SEQ ID NO: 3], Genscript). Aqueous solution 2: 8 mM di-cysteine modified metalloprotease-sensitive peptide crosslinker (MMP-sensitive crosslinker, Ac-GCRDGPQGIWGQDRCG-NH₂ [SEQ ID NO: 4]), Genscript), pre-reacted with 10 µM Alexa-fluor 568-maleimide (Life Technologies).

Both aqueous solutions were injected at the defined flow rates in a 1:1 volume mixture. Meanwhile, Novec 7500 Engineered Fluid (cat# 7100025016, 3 M) with 0.1% Pico-Surf™ (SF-000149, Sphere Fluidics) acting as a surfactant was used as the continuous oil phase, with the flow rate at 150 µl/mL. µGel beads were collected into a Corning centrifuge tube and cured at 37° C. for two hours. Thereafter, the cured µGel particles 12 were extracted and purified from the oil phase with a mixed solution of HEPES buffer (100 mM HEPES, 40 mM NaCl, pH 7.4) and hexane in a 1:1 volume, and centrifuged at 3000 rpm for five minutes at 4° C. The µGel pellets were further washed in HEPES buffer with 0.01% w/v Pluronic F-127 (Sigma) for five times to move the resident oil components. The µGel particles 12 in the aqueous solution was further allowed to swell and equilibrate with HEPES buffer at 4° C.

To make NPs 14 encapsulated in the µGel particles 12 (NPs-µGel), different amounts of NPs 14 (0, 25, 50, 100% weight percentage of NPs 14 to the weight of dry pre-gel components) was dispersed in aqueous solution 1. To enhance the uniform distribution of NPs 14 in the µGel particle 12 and stable droplet preparation, 0.25% (w/v) hyaluronic acid (HA700K, Lifecore Biomedical, LLC) was added in the dispersed particle solution.

For in vitro cell culture and in vivo evaluation, all µGel particles 12 (pure µGel, NPs-µGel, FR/therapeutic microporous hydrogel scaffold) were prepared with sterilized devices (PDMS device, connecting tubes) and sterile filtered pre-gel components by a 0.2 µm polyethersulfone membrane. All procedures were performed in a biosafety cabinet.

Size and swelling ratio: To determine the operational regime of droplet generation, at least five images of droplets in the channel were taken using a high-speed camera (Phantom) at each flow rate condition. The size distribution was analyzed by a custom-developed MATLAB code. The size of swollen µGel droplets in buffer solution was also measured in the same manner, and the volume swelling ratio was calculated by the following equation:

$${Q}_{v} = \frac{{d}_{a q} {}^{3}}{{d}_{o i l} {}^{3}}$$

where Qᵥ is the volume swelling ratio of a single droplet, d_{aq} is the diameter of droplets in the aqueous phase (HEPES buffer) and dₒᵢₗ is the diameter in the oil phase (Novec 7500).

NP loading concentration and efficiency in µGel: The NP loading concentration in µGel particles 12 was quantified by measuring fluorescent intensity of coumarin-labeled NPs 14. Briefly, the concentrated coumarin-labeled NPs-µGel particles 12 were diluted with HEPES buffer, and 100 µL solution was transferred to a 96-well plate to measure the fluorescent intensity (excitation: 485 nm, emission: 528 nm) by using a plate-reader. Meanwhile, the coumarin-labeled NPs 14 were diluted in HEPES buffer (0 to 8 mg/mL, 10 serial dilution points) to make the standard curve. The nanoparticle loading efficiency was calculated by the following equation:

$$\begin{matrix}\text{Particle loading efficiency (\%) = 100} \times \text{(Particle loading concentration} \\ \times \text{Swollen volume/Primary loading amount)} \text{.}\end{matrix}$$

Degradation of particle building blocks: To investigate the degradation and model drug release profiles of particles 12 or building blocks, NPs 14 were labeled by coumarin-6 dye and µGel particles 12 were labeled by AF546 dye. 100 µL of NPs-µGel particles 12 were added to the 1 mL PBS or collagenase II solutions (ranging from 1.6 mU/mL to 1 U/mL in PBS with calcium and magnesium) in centrifuge tubes and incubated at 37° C. with rotation at 20 rpm (n = 4 for each group). Three days later, spherical hydrogel particles 12 were centrifuged at 6000 rpm for 5 minutes, and 200 µL of the supernatant was transferred to a 96-well plate for measuring the release of coumarin-6 (excitation: 485 nm, emission: 528 nm) and AF546 (excitation: 556, emission: 573 nm) using a plate reader as surrogates for model drug release and hydrogel degradation, respectively. The µGel particles 12 were washed three times with PBS, and pushed through a 110 µm × 110 µm square microfluidic channel and imaged with fluorescence microscopy to measure the remaining model drug and AF546 as well as the deformability and swollen shape of the µGel particles 12.

Pore size and void fraction of MAP and therapeutic microporous hydrogel scaffolds: Fully swollen and equilibrated MAP or therapeutic spherical hydrogel particles 12 (20 µL) were activated by with 5 U/mL FXIIIa (Sigma) and 1 U/mL thrombin (Sigma), and the mixture was pipetted into a 3 mm diameter PDMS well on a glass coverslip, and annealed in a humidified incubator at 37° C. for 1.5 h to form porous MAP scaffolds or therapeutic microporous hydrogel scaffolds 10. Thereafter, the scaffolds were placed into HEPES buffer (pH 7.4) overnight to reach equilibrium. Samples were three-dimensional imaged using a Leica TCS SP8 confocal microscope with 10 × objective, spanning 1.16 mm × 1.16 mm (in x-and y-axis) × 200 µm (in z-axis). The pore size was analyzed by measuring the area (A) of voids within each confocal slice. Pore radius was calculated by an equation: Area (A)=π * radius². The pore size or diameter was equal to two times the radius.

Rheology properties: To determine the effects of particle loading amount on the gelation and gel rheology properties, rheological measurements were performed on bulk gel samples using a DHR-2 rheometer (TA Instruments). Briefly, different amount of PLGA35k/PLGA55k-b-PEG5k (50/50) NPs (0%, 25%, 50%, 100%, 200% of PEG weight) were quickly vortexed with two pre-gel aqueous solutions (basic aqueous solution 1 and 2 in 1:1 volume). To make a disk gel sample, a 40 µL mixed particle-containing solution was pipetted onto sterile slide glass siliconized with Sigmacote (SL2-25ML, Sigma-Aldrich), and covered with another glass slides with 1 mm spacer, followed with curing at 37° C. for two hours. Disc gels were swollen to equilibrium in HEPES buffer overnight before rheological measurements. A frequency sweep of 0.1-10 Hz was performed by using an 8-mm Peltier Plate-Crosshatched surface (TA Instruments), and the storage modulus and loss modulus were calculated from the average of the linear range. At least, four-disc gel samples were measured for each condition.

Drug release assay: Briefly, 2 mg of FR/NPs or 200 µl FR/therapeutic spherical hydrogel particles 12 were dispersed in a 0.22 µm filters inserted in a centrifuge tube (Corning™ Costar™ Spin-X™ Centrifuge Tube, Thermo Fisher Scientific) with 1 ml PBS (pH 7.4) at 37◦C, with continuous shaking. At discrete time intervals (16 hours, 1, 2, 4, 6 days), 0.5 ml of the sample solution was collected from the tube and frozen for the later analysis. Aliquots of the solutions were analyzed by reversed-phase separation and detection using tandem mass spectrometry with multiple reactions monitoring with previously optimized conditions for parent ion production and fragment ion detection on a triple quadrupole mass spectrometer (Agilent 6460). Quantification was achieved with the external standards for both analytes. All experimental samples were analyzed in triplicate and all results were reported as mean ± standard error of the mean.

Cellular uptake of NPs: To check cellular uptake of NPs 14 released from NPs-µGels, primary mice skin fibroblasts were seeded in 24-well plates at the density of 10,000 cells/cm2 and co-incubated with 0.1 mg coumarin-labeled NPs 14 or 20 µl NPs-µGel (50) beads (around the same weight of NPs) in the inserted Transwell (8 µm pore size), and cultured in the DMEM medium supplemented with 10% fetal bovine serum (FBS) and 1% penicillin/streptomycin (P/S). Cells were incubated in a humidified atmosphere containing 5% CO₂ at 37° C. Adhered cells were washed twice with PBS and fixed with 4% paraformaldehyde (PFA) on day 1 and day 4. The samples were stained with phalloidin F-actin and DAPI. The fluorescent images were taken by Zeiss Axio Observer Z1 inverted microscope and the fluorescent intensity was measured by Image J.

Cell isolation: Primary neonatal rat cardiomyocytes and fibroblasts were isolated from the hearts of 1-2 day old Sprague-Dawley rat pups as described previously with minor modifications. Briefly, the cardiac tissue was minced and digested with 80 units/mL collagenase II (Worthington) and 0.8 mg/mL pancreatin (Sigma) at 37° C. in a water bath. Neonatal calf serum (NCS) was applied to inactivate enzymatic activity in the digested cell mixture. The cell solution was filtered through 100 µm mesh and centrifuged at 2200 rpm for 3 minutes. The cell pellets were suspended in 1 mL NCS and further separated by Percoll density gradient centrifugation. A two-layer density gradient was formed consisting of 40.5% Percoll (GE17-0891-01, Sigma) solution in the top layer and 58.5% Percoll solution in the bottom layer. The cell suspension was layered on top of the gradient and centrifuged at 3000 rpm at room temperature for 30 minutes. Fibroblasts equilibrated and collected form the top of the transparent Percoll solution. Cardiomyocytes could subsequently be removed from the newly formed layer between the Percoll solutions and harvested separately. Both cells were washed with warm DMEM medium containing 10% FBS and 1% P/S and used immediately.

Cell culture and evaluation of drug effects in vitro: The drug effects on cardiomyocyte viability and proliferation were evaluated. Isolated cardiomyocytes were calculated and seeded on 0.1% gelatin-coated twenty-four well tissue culture plate with a density of 20, 000 cells/cm², and cultured at 37° C. in a humidified, 5% CO₂ incubator overnight in DMEM/Medium 199 (4/1) containing 10% FBS, 1% NEAAs and 1% P/S. The next day, the culture medium was replaced by fresh medium containing 20 µM F, R or their combination. The medium was changed every other day. Cell viability assay and proliferation assay of cardiomyocytes were performed at day 1, 3 and 5. A live/dead kit (Invitrogen) was for cell viability assay, and images were taken using inverted microscope fluorescence microscopy (Zeiss Axio Observer Z1) to determine the cell numbers and the percentage of dead cells. To analyze the proliferation of cardiomyocyte, cells were stained by the Click-iT® EdU assay (Invitrogen) as the vendor-provided protocol. Briefly, cells treated with EdU concentration of 10 µM for 24 hours before fixing with 4% PFA in PBS, followed with EdU detection and immunofluorescent staining with cTnT antibody (DSHB).

The drug effects on cardiac fibroblast proliferation and myo-differentiation were evaluated. Fibroblasts were seeded on twenty-four well tissue culture plate with a density of 5000 cells/cm² and cultured overnight in DMEM containing 10% FBS and 1% P/S. On the next day, the culture medium was replaced by the fresh medium containing F and R at the determined concentrations and combinations, and the medium was changed every other day. MTS cell proliferation assay (cat# PR-G3582, Thermo Fisher Scientific) was performed on day 1, 3 and 5 by following the protocol from the manufacturer. Meanwhile, some cells were fixed with 4% PFA for myo-differentiation assay by fluorescent staining using α-SMA antibody (Abcam) and phalloidin (for F-actin) (Thermo Fisher Scientific).

The drug effects on EC proliferation and network formation were evaluated. Human umbilical vein endothelial cells (HUVECs) were seeded on 0.1% gelatin-coated 24-well plates with a density of 5, 000 cells/cm² and cultured overnight in DMEM containing 10% FBS and 1% P/S. On the next day, the culture medium was replaced by the fresh medium containing F and R at the determined concentrations and combinations, and the medium was changed every other day. MTS cell proliferation assay was performed on day 1, 3 and 5. In addition, the ECs network formation was examined on growth factor reduced Matrigel according to the manufacturer’s instructions (cat# CB-40230C, Thermo Fisher Scientific). Briefly, 24-well plates were coated with Matrigel. ECs were digested and plated onto a layer of Matrigel at a density of 3×10⁵ cells/well in M199 medium containing 1% FBS and 1% P/S, with the addition of F and R. Vascular endothelial growth factor (VEGF, 20 ng/mL) was used as a positive control. After 16 hours of culture, cells were stained with calcein acetoxymethyl ester (calcein-AM) and observed with an inverted fluorescent microscope. The number of tubular structures, junctions and meshes were analyzed by Image J with the Angiogenesis Analyzer plugin (n = 4-6 per group).

The effects of drugs released from FR/NPs and FR/NPs-µGel particles 12 were evaluated using the methods mentioned above. During cell culture, 2 mg FR/NPs or 100 µl FR/NPs-µGel (100) (theoretical loading weight of NPs ~2 mg) was added in the inserted Transwell (0.4 µm pore size) in 24-well plates with cultured cells.

MI model and intramyocardial injection of nanoparticle containing µGel particles: All animal work was conducted under protocols approved by the University of California Los Angeles (#2016-101-11) and the University of California San Francisco (#AN176681-02) and was performed in accordance with the recommendations of the American Association for Accreditation of Laboratory Animal Care. The ischemia-reperfusion MI model was established as previously described in L. V. Le et al., Injectable hyaluronic acid based microrods provide local micromechanical and biochemical cues to attenuate cardiac fibrosis after myocardial infarction, Biomaterials 2018 Jul;169:11-21, which is incorporated herein by reference. Briefly, the left anterior descending coronary artery of female Sprague-Dawley rats (200-250 g, 8-10 weeks) underwent ligation for 30 minutes, followed by reperfusion. The intramyocardial injections (50 µL, twice) of sterile PBS, FR/NPs (20 mg/mL in PBS), MAP gel and FR/therapeutic microporous hydrogel (that formed the scaffold) were performed 2 days post-MI via ultrasound-guided transthoracic injection using a 27-gauge syringe. The successful injection was confirmed by a slight local increase of ultrasound signal in the LV wall.

Echocardiographic assessment: Echocardiography was performed at 2-day post-MI and five weeks post-injection using standard methods. Transthoracic echocardiography was performed with a 15-MHz linear array transducer system (Sequoia c256, Acuson, Erlangen, Germany) on all animals anesthetized with isoflurane. The left ventricular end-diastolic volume (LVEDV), left ventricular end-systolic volume (LVESV) and ejection fraction (LVEF) were measured. All measurements were the averages of three consecutive cardiac cycles. Cases where ejection fraction was above 45% at day 2 were excluded from echocardiographic and histological analyses because they indicated an insufficient infarct model. Finally, PBS (n = 9), FR/NPs (n = 6), MAP gel (n = 9) and FR/drugMAP gel (n = 9) were applied for all in vivo characterizations.

Histology and immunostaining: At 5 weeks after the injection, all rats were sacrificed for tissue harvesting. The hearts were embedded in optimal cutting temperature (OCT) compound, fresh frozen by dry ice immediately, and stored at -80° C. All tissue blocks were cryosectioned at a thickness of 10 µm by a cryostat microtome (HM525 NX, Thermo Fisher Scientific) starting from the apex of the left ventricle, and 10 serial sections were collected for every 500 µm intervals. All slides were kept at -20° C. for later staining.

Sections were stained with Masson’s trichrome staining using standard protocols and images were captured with an inverted microscope (Nikon, Eclipse Ti-S fluorescence microscope). Masson’s-trichrome staining images were used to evaluate the infarct size, fibrosis area and LV wall thickness with Image J software. The infarct size or scar area (% LV) was calculated by dividing the collagen deposited area to the entire left ventricle area. LV wall thickness was calculated by averaging the minimum infarcted LV wall thickness of all samples for each group.

For immunofluorescent staining, cell samples or air-dried tissue slides were fixed with 4% PFA for 10 minutes at room temperature, permeabilized with 0.1% Triton™ X-100 for 15 minutes, and blocked with 10% normal goat serum for 1 hour at room temperature. The cell samples and slides were incubated with primary antibodies, against α-SMA (rabbit, Abcam, ab5694, 1:300), Von Willebrand Factor (vWF, sheep, Abcam, ab11713), CD68 (mouse, Abcam, ab955, 1:300), cardiac Troponin T (cTnT, mouse, DSHB, 1:200) or Ki67 (Rabbit, Abcam, ab16667, 1:200) overnight at 4° C. Thereafter, appropriate Alexafluor 488-or Alexafluor 546- or Alexa fluor 637-conjugated secondary antibodies (Thermo Fisher Scientific) was added and incubated for 1 hour at room temperature. Thereafter, nuclei were stained with 4′,6-diamindino-2-phenylindole (DAPI, 1:2,500 in sterilized deionized water, Sigma) for 10 minutes in the dark. All fluorescent images were taken with Zeiss Axio Observer Z1 inverted microscope and confocal Inverted Leica TCS-SP8-SMD Confocal Microscope.

### Statistical Analysis

Data are presented as means ± standard deviations, calculated from the average of at least three biological replicates unless otherwise specified. Statistical analysis was performed using one-way analysis of variance (ANOVA), followed by post-hoc analysis with Turkey’s test using Origin 8 software. P values < 0.05 were considered statistically significant.

While embodiments of the present invention have been shown and described, various modifications may be made without departing from the scope of the present invention. For example, the NPs may be loaded with different drugs or therapeutic agents other than F and/or R. In some embodiments only a single drug or therapeutic agent may be loaded therein. Further, the NPs may be made from different materials other than PLGA/PEG in other embodiments. Likewise, the therapeutic hydrogel may be used to treat other tissues types and for different applications including but not limited to tissue repair, tissue regeneration, cancer therapy, and immunotherapy. The invention, therefore, should not be limited, except to the following claims, and their equivalents.

What is claimed is:

1. A therapeutic hydrogel system for use in an animal comprising:
a plurality of spherical hydrogel particles decorated with K peptides and Q peptides and having distributed therein a plurality of nanoparticles loaded with one or more therapeutic agents or drugs, wherein the plurality of spherical hydrogel particles when exposed to an annealing agent, induces surface binding between the spherical hydrogel particles via the K peptides and Q peptides and forms a porous scaffold.

2. The therapeutic hydrogel system of claim 1, wherein the one or more therapeutic agents or drugs comprises one of: a small molecule, a cytokine, a protein or fragment thereof, a peptide, a vaccine, a nucleic acid, a gene or a genetic sequence, and a biomolecule.

3. The therapeutic hydrogel system of claim 1, wherein the one or more therapeutic agents or drugs comprises a cAMP agonist and/or a TGF-β inhibitor.

4. The therapeutic hydrogel system of claim 1, wherein the one or more therapeutic agents or drugs comprises Forskolin and 2-(3-(6-Methylpyridine-2-yl)-1H-pyrazol-4-yl)-1,5-naphthyridine (RepSox).

5. The therapeutic hydrogel system of claim 1, wherein the plurality of nanoparticles comprise poly(lactic-co-glycolic acid) (PLGA) nanoparticles.

6. The therapeutic hydrogel system of claim 1, wherein the plurality of nanoparticles comprise poly(lactic-co-glycolic acid) (PLGA)-polyethlyene glycol (PEG) nanoparticles.

7. The therapeutic hydrogel system of claim 6, wherein the plurality of nanoparticles comprise PLGA55k-b-PEG5k.

8. The therapeutic hydrogel system of claim 1, wherein the plurality of spherical hydrogel particles are decorated with RGD peptide.

9. The therapeutic hydrogel system of claim 1, wherein the spherical hydrogel particles are biodegradable via a MMP-sensitive crosslinker.

10. The therapeutic hydrogel system of claim 1, wherein the nanoparticles comprise PLGA35k/PLGA55k-PEG5k in a weight ratio of substantially 1:1.

11. A therapeutic hydrogel system for use in an animal comprising:
a plurality of spherical hydrogel particles having distributed therein a plurality of nanoparticles loaded with one or more therapeutic agents or drugs, wherein the plurality of spherical hydrogel particles when exposed to an annealing agent, induces surface binding between the spherical hydrogel particles and forms a porous scaffold.

12. The therapeutic hydrogel system of claim 11, wherein the system further comprises an exogenous annealing agent.

13. The therapeutic hydrogel system of claim 11, wherein an annealing moiety on the surface of the hydrogel particles links adjacent hydrogel particles.

14. A method of manufacturing a therapeutic hydrogel comprising:
providing a microfluidic device configured to generate aqueous emulsions in an oil phase, the microfluidic device having a first aqueous phase microfluidic channel and a second aqueous phase microfluidic channel interfacing in an emulsion generating region, the emulsion generating region interfacing with a first oil phase microfluidic channel and a second oil phase microfluidic channel;
flowing a first aqueous solution into the first aqueous phase microfluidic channel comprising PEG vinyl sulfone pre-reacted with K-peptide, Q-peptide, RGD peptide, and a plurality of nanoparticles loaded with one or more therapeutic agents or drugs;
flowing a second aqueous solution into the second aqueous phase microfluidic channel comprising a MMP-sensitive crosslinker;
flowing oil into the first and second oil phase microfluidic channels to generate emulsions of the mixed first and second aqueous solutions in the oil; and
allowing the emulsions to crosslink to form spherical hydrogel particles.

15. The method of claim 14, wherein the first aqueous solution further comprises a cell adhesive moiety.

16. The method of claim 15, wherein the cell adhesive moiety comprises RGD peptide.

17. The method of claim 14, wherein the first aqueous solution contains less than about 1% v/v% hyaluronic acid (HA).

18. The method of claim 14, wherein the plurality of nanoparticles comprise poly(lactic-co-glycolic acid) (PLGA) nanoparticles.

19. The method of claim 14, wherein the plurality of nanoparticles comprise poly(lactic-co-glycolic acid) (PLGA)-polyethlyene glycol (PEG) nanoparticles.

20. The method of claim 19, wherein the plurality of nanoparticles comprise PLGA55k-b-PEG5k.

21. The method of claim 14, wherein the one or more therapeutic agents or drugs comprises a cAMP agonist and/or a TGF-β inhibitor.

22. The method of claim 14, wherein the one or more therapeutic agents or drugs comprises Forskolin and 2-(3-(6-Methylpyridine-2-yl)-1 H-pyrazol-4-yl)-1,5-naphthyridine RepSox.

23. A method of treating myocardial infarction in an animal comprising:
injecting a therapeutic hydrogel system into heart tissue of the animal, the therapeutic hydrogel system comprising a plurality of spherical hydrogel particles decorated with K peptides and Q peptides and having distributed therein a plurality of nanoparticles loaded with one or more therapeutic agents or drugs, wherein the plurality of spherical hydrogel particles when exposed to an annealing agent, induces surface binding between the spherical hydrogel particles via the K peptides and Q peptides and forms a porous scaffold.

24. The method of claim 23, wherein the one or more therapeutic agents or drugs comprises a cAMP agonist and/or a TGF-β inhibitor.

25. The method of claim 23, wherein the one or more therapeutic agents or drugs comprises Forskolin and 2-(3-(6-Methylpyridine-2-yl)-1H-pyrazol-4-yl)-1,5-naphthyridine RepSox.

26. The method of claim 23, wherein the plurality of nanoparticles comprise poly(lactic-co-glycolic acid) (PLGA) nanoparticles.

27. The method of claim 23, wherein the plurality of nanoparticles comprise poly(lactic-co-glycolic acid) (PLGA)-polyethlyene glycol (PEG) nanoparticles.

28. The method of claim 23, wherein the plurality of nanoparticles comprise PLGA55k-b-PEG5k.

29. The method of claim 23, wherein the therapeutic hydrogel is injected into an interior or exterior surface of heart tissue.

30. (canceled)


SEQUENCE LISTING (4 sequences)

SEQ ID NO: 1 - protein, 8 aa, Artificial Sequence
  Linker molecule
FKGGERCG

SEQ ID NO: 2 - protein, 14 aa, Artificial Sequence
  Linker molecule
NQEQVSPLGGERCG

SEQ ID NO: 3 - protein, 10 aa, Artificial Sequence
  Cell adhesive peptide
RGDSPGERCG

SEQ ID NO: 4 - protein, 16 aa, Artificial Sequence
  matrix metalloprotease degradable crosslinker
GCRDGPQGIWGQDRCG